(19) United States
(12) Patent Application Publication
(10) Pub. No.: US 2023/0381082 A1
(43) Pub. Date: Nov. 30, 2023

(54) PHOTOPROTECTIVE COSMETIC COMPOSITIONS COMPRISING XANTHOMMATIN AS AN ANTIOXIDANT AND UV-FILTER STABILIZER

(71) Applicant: SEASPIRE, INC., Cambridge, MA (US)

(72) Inventors: Leila F. Deravi, Cambridge, MA (US); Camille A. Martin, Cambridge, MA (US); Natalie Cox, Cambridge, MA (US)

(21) Appl. No.: 18/031,916

(22) PCT Filed: Oct. 15, 2021
(86) PCT No.: PCT/US2021/055128

§ 371 Date: Apr. 14, 2023

Related U.S. Application Data
(60) Provisional application No. 63/092,851, filed on Oct. 16, 2020.

(51) Int. Cl. A61K 8/49; A61K 8/35; A61K 8/37; A61K 8/40; A61Q 17/04; A61K 8/29; A61K 8/27

(52) U.S. Cl. CPC A61K 8/4926; A61K 8/35; A61K 8/37; A61K 8/40; A61Q 17/04; A61K 8/29; A61K 8/27; A61K 8/4973; A61K 8/498; A61K 2800/522

(57) ABSTRACT

Unsaturated solutions of phenoxazone and/or phenoxazine small molecules, or precursors or derivatives thereof, for example xanthommatin, uses the phenoxazone and/or phenoxazine as standalone discrete molecules in dermatological and skin care formulations to enhance protection and safety. The unaggregated xanthommatin molecules are capable of boosting UV-filtering performance and increasing the photo-stability and UV-absorptive properties of known chemical UV filters. Accordingly, unaggregated xanthommatin in an unsaturated solution can replace the performance of the UV-A filter avobenzone in suncare products and replace the performance of known antioxidants producing more performance stability over time. By integration of unsaturated solutions of xanthommatin molecules into dermatological formulations, and in particular into sun protecting formulations such as sunscreen and facial moisturizer, enhanced protection can be achieved without the systemic toxicities associated with traditional chemical and physical UV filters. Unsaturated solutions of xanthommatin provide safer, more effective, and longer-lasting broad spectrum protection in sun screen formulations.

### RELATED APPLICATIONS

This application claims the benefit of priority to U.S. Provisional Patent Application No. 63/092,851, filed Oct. 16, 2020, which application is hereby incorporated by reference in their entirety.

### FUNDING

This invention was made with government support under Grant NSF SBIR 2050284 awarded by the National Science Foundation. The government has certain rights in the invention.

### BACKGROUND

Environmental exposure to solar ultraviolet (UV) radiation remains one of the main contributors to nearly 3.5 million cases of skin cancer each year. With few effective therapeutic options available for melanoma, a significant effort has been directed towards preventative skin care through the daily application of topical sunscreens.

However, a recent report by the Environmental Working Group (EWG) described that >70% of the UV filters in sunscreens are either (1) not effective at protecting human skin from UV-radiation and/or (2) contain toxic components. Together, with accumulating data supporting the onset of developmental and reproductive toxicities in animals, these data indicate that chronic use of commercial sunscreens is an emerging threat to both human and marine health, forcing the industry along with the U.S. Food and Drug Administration (FDA) to re-evaluate current over-the-counter (OTC) sunscreen offerings.

Known antioxidants (vitamin C and E) are unstable and readily react in air and with sunlight to form byproducts that are not effective (and sometimes toxic). Phenoxazone and phenoxazine are small molecules that can be used as sun protective formulations. For example, aggregated xanthommatin in a stabilized three-dimensional form has been incorporated into suncare formulations. The xanthommatin particle aggregates used in these formulations are typically greater than 100 nm in size.

There is a critical need to develop safer, ecofriendly, and more effective UV-filters to replace those currently in use.

Growing trends support the application and utility of biologically derived or biologically inspired raw materials in cosmetics, as the industry continues to push towards natural and eco-friendly product offerings designed to replace many non-sustainable and sometimes toxic counterparts. Bom, S., et al., A step forward on sustainability in the cosmetics industry: A review. Journal of Cleaner Production, 2019. 225: p. 270-290. Bom, S., et al., Replacing Synthetic Ingredients by Sustainable Natural Alternatives: A Case Study Using Topical O/W Emulsions. Molecules, 2020. 25(21). Despite the origin of their inspiration or sourcing, these bio-based or bio-inspired raw materials are still subject to rigorous safety screening and standards before they can be incorporated in over-the-counter products Bom, 2019. Bom, S., H. M. Ribeiro, and J. Marto, Sustainability Calculator: A Tool to Assess Sustainability in Cosmetic Products. Sustainability, 2020. 12(4): p. 1437. Ammonium xanthommatin (referred to hereon as xanthommatin) is a naturally occurring biochrome present in arthropods and cephalopods and is formed during the metabolism of tryptophan in these species. Riou, M. and J. P. Christides, Cryptic color change in a crab spider (Misumena vatia): identification and quantification of precursors and ommochrome pigments by HPLC. J Chem Ecol, 2010. 36(4): p. 412-23. Futahashi, R. a. K., Ryoji and Mano, Hiroaki and Fukatsu, Takema, Redox alters yellow dragonflies into red. PNAS 2012. 109(31): p. 12626-12631. Osani-Futahashi, M., et al., Positional cloning of a Bombyz pink-eyed white egg locus reveals the major role of cardinal in ommochrome synthesis. Heredity, 2016. 116: p. 135-145. Williams, T. L., et al., Dynamic pigmentary and structural coloration within cephalopod chromatophore organs. Nature communications, 2019. 10. Deravi, L. F., et al., The structure-function relationships of a natural nanoscale photonic device in cuttlefish chromatophores. J R Soc Interface, 2014. 11(93): p. 20130942. Williams, T. L., et al., Contributions of phenoxazone-based pigments to the structure and function of nanostructured granules in squid chromatophores. Langmuir, 2016. 32(15): p. 3754-3759. Williams, T. L., et al., Dynamic pigmentary and structural coloration within cephalopod chromatophore organs. Nat Commun, 2019. 10(1): p. 1004.

### SUMMARY

Improved compositions are disclosed herein for suncare and cosmetic applications. The new compositions include unsaturated solutions of one or more compounds from the bioinspired, photo-stable class of phenoxazone and phenoxazine (or derivatives or precursors thereof), to provide for more effective use in providing sun and cosmetic care. As used herein, an unsaturated solution of one or more phenoxazone and/or phenoxazine compound includes chemical solutions in which the one or more phenoxazone and/or phenoxazine compound is fully dissolved in solution. Though there may be ionic bonding, electrostatic bonding, hydrogen bonding, and Van der Waals bonding between the small molecules in the solution and other solution components, the one or more phenoxazone and/or phenoxazine compound in the unsaturated solution are not bound to other active molecular constituents by covalent or other bonds. The one or more phenoxazone and/or phenoxazine compound in the unsaturated solution may also be bound to inactive side chains and/or polymers for enhanced stabilization in dermatological or other formulations, but are not bound or affiliated with active molecules of the same or different species. The one or more phenoxazone and/or phenoxazine compound in the unsaturated solution are aggregated and do not form three-dimensional stabilized structures with other phenoxazone and/or phenoxazine compound or other active molecules in the solution or greater formulation. The unsaturated solution of one or more phenoxazone and/or phenoxazine compound may include unsaturated solutions of phenoxazone and/or phenoxazine derivatives or precursors, including 3-hydroxykynurenine. The unsaturated solution of one or more phenoxazone and/or phenoxazine compound may also include xanthommatin, decarboxylated xanthommatin, uncyclized xanthommatin, ommatin D, dihydroxy-xanthommatin, or rhodommatin, a derivative of any of the foregoing, a precursor of any of the foregoing, and salts of any of the foregoing. In certain embodiments, the phenoxazone and/or phenoxazine compound, or a salt thereof, is xanthommatin, or a salt thereof. In certain such embodiments, the phenoxazone and/or phenoxazine compound, or a salt thereof, is ammonium xanthommatin.

The unsaturated solutions of one or more phenoxazone and/or phenoxazine compound (e.g., xanthommatin, or a salt thereof, such as ammonium xanthommatin) provide the composition with stabilizing and boosting properties relative to bound phenoxazone and phenoxazine compounds, because the molecular and chemical properties of the unaggregated small molecules in the unsaturated solution can be harnessed in UV protective formulations. Unsaturated solutions of one or more phenoxazone and/or phenoxazine compound are easily incorporated in a formulation and the chemistry of the unaggregated small molecules can be harnessed to provide surprising properties with regard to UV-absorption, boosting, and stabilizing. In certain adaptations, the unsaturated solution of one or more phenoxazone and/or phenoxazine compound comprises xanthommatin, one or more derivatives or precursors thereof, or salts thereof. Xanthommatin is a biochrome present in anthropods and cephalopods but may be synthetically formed for the compositions disclosed herein.

The present application provides a composition comprising at least one UV-filtering material (e.g., a UV-absorbing compound or a UV-scattering particle) and an unsaturated solution of 1 wt % or less of one or more phenoxazone and/or phenoxazine compound, or a precursor or a derivative thereof, or a salt of any of the foregoing. In certain such embodiments, the unsaturated solution of 1 wt % or less of one or more phenoxazone and/or phenoxazine compound, or a precursor or a derivative thereof, or a salt of any of the foregoing comprises xanthommatin, decarboxylated xanthommatin, uncyclized xanthommatin, ommatin D, dihydroxy-xanthommatin, rhodommatin, a derivative or precursor thereof, or a salt of any of the foregoing. In certain embodiments, the unsaturated solution of 1 wt % or less of one or more phenoxazone and/or phenoxazine compound, or a precursor or a derivative thereof, or a salt of any of the foregoing comprises 3-hydroxykynurenine. In other embodiments, the unsaturated solution of 1 wt % or less of one or more phenoxazone and/or phenoxazine compound, or a precursor or a derivative thereof, or a salt of any of the foregoing comprises xanthommatin, one or more derivatives or precursors thereof, or salts thereof (e.g., ammonium xanthommatin). In certain embodiments, the unsaturated solution of 1 wt % or less of one or more phenoxazone and/or phenoxazine compound, or a precursor or a derivative thereof, or a salt of any of the foregoing, comprises synthetic molecules. In certain embodiments, the unsaturated solution of 1 wt % or less of one or more phenoxazone and/or phenoxazine compound, or a precursor or a derivative thereof, or a salt of any of the foregoing, comprises unaggregated molecules.

In certain embodiments, the unsaturated solution of one or more phenoxazone and/or phenoxazine compound, or a precursor or a derivative thereof, of the present application provides a UV-filter which can absorb up to 3×more across the broader UV-visible spectrum than conventional chemical filters.

In certain embodiments of the compositions of the present application, the at least one UV-filtering material (e.g., a UV-absorbing compound or a UV-scattering particle) and the unsaturated solution of 1 wt % or less of one or more phenoxazone and/or phenoxazine compound, a precursor or derivative thereof, or a salt of any of the foregoing together exhibit synergy. In certain embodiments, the composition has a pre-irradiation SPF greater than a composition comprising the at least one UV-filtering material (e.g., a UV-absorbing compound or a UV-scattering particle) without the unsaturated solution of 1 wt % or less of one or more phenoxazone and/or phenoxazine compound, a precursor or derivative thereof, or a salt of any of the foregoing. In certain embodiments, the composition has a post-irradiation SPF greater than a composition comprising the at least one UV-filtering material (e.g., a UV-absorbing compound or a UV-scattering particle) without the unsaturated solution of 1 wt % or less of one or more phenoxazone and/or phenoxazine compound, a precursor or derivative thereof, or a salt of any of the foregoing. In certain embodiments, the composition has an increase in SPF of greater than 10% as compared to a composition comprising the at least one UV-filtering material (e.g., a UV-absorbing compound or a UV-scattering particle) without the unsaturated solution of 1 wt % or less of one or more phenoxazone and/or phenoxazine compound, a precursor or derivative thereof, or a salt of any of the foregoing. In certain embodiments, the composition has an increase in SPF of greater than 25% as compared to a composition comprising the at least one UV-filtering material (e.g., a UV-absorbing compound or a UV-scattering particle) without the unsaturated solution of 1 wt % or less of one or more phenoxazone and/or phenoxazine compound, a precursor or derivative thereof, or a salt of any of the foregoing. In certain embodiments of the foregoing, the SPF of the composition is maintained for at least one week. In certain embodiments of the foregoing, the SPF of the composition is maintained for at least two weeks. In certain embodiments of the foregoing, the SPF of the composition is maintained for at least three weeks.

In certain embodiments of the compositions of the present application, the composition exhibits greater UV absorbance than a composition comprising the at least one UV-absorbing compound without the unsaturated solution of 1 wt % or less of one or more phenoxazone and/or phenoxazine compound, a precursor or derivative thereof, or a salt of any of the foregoing. In certain embodiments, the composition exhibits a change in UV absorbance of greater than 100% as compared to a composition comprising the at least one UV-absorbing compound without the unsaturated solution of 1 wt % or less of one or more phenoxazone and/or phenoxazine compound, a precursor or derivative thereof, or a salt of any of the foregoing. In certain embodiments, the composition exhibits a change in UV absorbance of greater than 150% as compared to a composition comprising the at least one UV-absorbing compound without the unsaturated solution of 1 wt % or less of one or more phenoxazone and/or phenoxazine compound, a precursor or derivative thereof, or a salt of any of the foregoing. In certain embodiments, the composition exhibits a change in UV absorbance of greater than 200% as compared to a composition comprising the at least one UV-absorbing compound without the unsaturated solution of 1 wt % or less of one or more phenoxazone and/or phenoxazine compound, a precursor or derivative thereof, or a salt of any of the foregoing.

In certain embodiments, the compositions of the present application comprising at least one UV-filtering material (e.g., a UV-absorbing compound or a UV-scattering particle) and an unsaturated solution of 1 wt % or less of one or more phenoxazone and/or phenoxazine compound, or a precursor or a derivative thereof, may be formulated as a solution. In certain such embodiments, the at least one UV-filtering material (e.g., a UV-absorbing compound or a UV-scattering particle) and the unsaturated solution of 1 wt % or less of one or more phenoxazone and/or phenoxazine compound, a precursor or derivative thereof, or a salt of any of the foregoing are homogeneously distributed within the solution. In other embodiments, the compositions of the present application comprising at least one UV-filtering material (e.g., a UV-absorbing compound or a UV-scattering particle) and an unsaturated solution of 1 wt % or less of one or more phenoxazone and/or phenoxazine compound, or a precursor or a derivative thereof, may be formulated as an emulsion. In certain such embodiments, the unsaturated solution of one or more phenoxazone and/or phenoxazine compound, or a precursor or a derivative thereof comprises a soluble portion of the solution phase of the emulsion.

In certain embodiments, the compositions of the present application comprising at least one UV-filtering material (e.g., a UV-absorbing compound or a UV-scattering particle) and an unsaturated solution of 1 wt % or less of one or more phenoxazone and/or phenoxazine compound, a precursor or derivative thereof, or a salt of any of the foregoing, may be formulated as a cream, gel, spray, or lotion, for use in a cosmetic or dermatological formulation. In certain embodiments, the composition of the present application is formulated to provide protection from solar ultraviolet radiation. In certain embodiments, the composition of the present application is formulated to provide broad spectrum protection to protect against blue light and infrared regions. In certain embodiments, the composition of the present application is formulated to provide a ratio of UVA I to UV B filters of at least 0.7 or greater. Unsaturated solutions of one or more phenoxazone and/or phenoxazine compound, or a precursor or a derivative thereof (e.g., xanthommatin, derivatives or precursors thereof, or a salt of any of the foregoing, such as ammonium xanthommatin) can absorb and scatter a broad range (UV-near IR) of sunlight to provide enhanced activity as a skin protectant. In certain embodiments, the composition is formulated to provide a sun protection factor (“SPF”) of at least 15, 30, 60 or 100. In certain embodiments, the composition is formulated to provide a SPF of 15-100. In certain embodiments, the composition is formulated to provide a UVA protection factor (“UVA-PF”) of at least 15, 30, 60, or 100. In certain embodiments, the composition is formulated to provide a UVA-PF of 15-100. In certain embodiments, the composition is formulated to provide a ratio of UVA I to UV filters of at least 0.7 or greater.

In certain embodiments, the composition of the present application further comprises one or more non-ionic polymeric emulsifier. In certain such embodiments, the one or more non-ionic polymeric emulsifier is selected from potassium cetyl phosphate, PEG-150 distearate, cetearyl alcohol, caprylic/capric triglyceride, and glyceryl stearate.

In certain embodiments, the composition of the present application further comprises at least one additional anti-oxidizing compound. In certain such embodiments, the at least one additional anti-oxidizing compound comprises one or more of arbutin, BHA, BHT, koji acid, hydroxyanisole, hydroquinone, t-butyl hydroquinone, tocopherol, nordihydroguaiaretic acid, rosmarinic acid, Trolox, goosypol, flavone, flavanone, isoflavones, flavanol, protocatechuic acid, resorcylic acid, gallic, caffeic acid, ferulic acid, chlorogenic acid, ascorbic acid, ascorbyl palmitate, carotenoids, cysteine hydrochloride, dithiothreitol, glutathione, thioglycolic acid, thiodipropionic acid, alpha-lipoic acid, and/or xanthines. In certain embodiments, the unsaturated solution of one or more phenoxazone and/or phenoxazine compound, a precursor or derivative thereof, or a salt of any of the foregoing (e.g., xanthommatin, a derivative or precursor thereof, or a salt of any one of the foregoing, such as ammonium xanthommatin), functions as an anti-oxidizing compound in the composition. In certain embodiments, the at least one additional anti-oxidizing compound is present at 0.1-5 wt %. In certain embodiments, the at least one additional anti-oxidizing compound is present at 0.1-1 wt %.

In certain embodiments of the compositions of the present application, the composition of the present application further comprises one or more anti-radical compound. In certain such embodiments, the anti-radical compound is present in the final formulation in an amount of about 0.1-15 wt %, 0.1-1 wt %, 1-10 wt %, or 6-8 wt %. In certain embodiments, the anti-radical compound is present in the final formulation in an amount of about 0.1 wt %, 0.5 wt %, 1 wt %, 6 wt %, 8 wt %, 10 wt %, 15 wt %, or any other suitable amount.

In certain embodiments of the compositions of the present application, the at least one UV-filtering material (e.g., a UV-absorbing compound or a UV-scattering particle) is present in an amount of 0.1-15 wt %, 0.1-5 wt %, 0.1-1 wt %. In certain embodiments, the at least one UV-filtering material (e.g., a UV-absorbing compound or a UV-scattering particle) is present in an amount of 0.1 wt %, 0.5 wt %, 1 wt %, 6 wt %, 8 wt %, 10 wt %, 15 wt %, or any other suitable amount. In certain embodiments, the UV-filtering material (e.g., a UV-absorbing compound or a UV-scattering particle) comprises an FDA approved UV-filter or another UV-filtering compound. In certain such embodiments, the UV-absorbing compound comprises one or more of avobenzone, oxybenzone, oxybenzone cinoxate, homosalate, octisalate, octinoxate, octocrylene, and/or trolamine salicylate. In other embodiments, the UV-scattering particle comprises titanium dioxide or zinc dioxide.

In certain embodiments of the compositions of the present application, the composition comprises 0.1-1 wt % phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin). In certain embodiments of the compositions of the present application, the composition comprises 0.01-0.1 wt % phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin). In certain embodiments of the compositions of the present application, the composition comprises 0.01-0.05 wt % phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin). In certain embodiments, the unsaturated solution of one or more phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin), is present in the final formulation in an amount of about 0.01 wt %, 0.03 wt %, 0.05 wt %, 0.1 wt %, 0.5 wt %, or 1 wt %.

The present application provides a method of maintaining the SPF of a composition comprising at least one UV-filtering material (e.g., a UV-absorbing compound or a UV-scattering particle), comprising adding an unsaturated solution comprising 1 wt % or less of one or more phenoxazone and/or phenoxazine compound, a precursor or derivative thereof, or a salt of any of the foregoing to the composition to provide a final cosmetic formulation. In certain embodiments, the SPF is maintained for at least one week, for at least two weeks, or for at least three weeks.

The present application further provide a method of increasing the SPF of a composition comprising at least one UV-filtering material (e.g., a UV-absorbing compound or a UV-scattering particle), comprising adding an unsaturated solution comprising 1 wt % or less of one or more phenoxazone and/or phenoxazine compound, a precursor or derivative thereof, or a salt of any of the foregoing to the composition to provide a final cosmetic formulation. In certain embodiments, the at least one UV-filtering material (e.g., a UV-absorbing compound or a UV-scattering particle) and the unsaturated solution of 1 wt % or less of one or more phenoxazone and/or phenoxazine compound, a precursor or derivative thereof, or a salt of any of the foregoing together exhibit synergy. In certain embodiments, the composition has an increase in pre-irradiation SPF of greater than 10% as compared to a composition comprising the at least one UV-filtering material (e.g., a UV-absorbing compound or a UV-scattering particle) without the unsaturated solution of 1 wt % or less of one or more phenoxazone and/or phenoxazine compound, a precursor or derivative thereof, or a salt of any of the foregoing. In certain embodiments, the composition has an increase in post-irradiation SPF of greater than 10% as compared to a composition comprising the at least one UV-filtering material (e.g., a UV-absorbing compound or a UV-scattering particle) without the unsaturated solution of 1 wt % or less of one or more phenoxazone and/or phenoxazine compound, a precursor or derivative thereof, or a salt of any of the foregoing.

In certain embodiments of the methods of the present application, the one or more phenoxazone and/or phenoxazine compound, a precursor or derivative thereof, or a salt of any of the foregoing comprises an xanthommatin, decarboxylated xanthommatin, uncyclized xanthommatin, ommatin D, dihydroxy-xanthommatin, or rhodommatin, or a precursor or derivative thereof, or a salt of any of the foregoing. In certain such embodiments, the one or more phenoxazone and/or phenoxazine compound, a precursor or derivative thereof, or a salt of any of the foregoing comprises xanthommatin, or salt thereof. In certain embodiments of the methods of the present application, the one or more phenoxazone and/or phenoxazine compound, a precursor or derivative thereof, or a salt of any of the foregoing comprises between 0.01-0.1 wt % in the final cosmetic formulation. In certain embodiments of the methods of the present application, the one or more phenoxazone and/or phenoxazine compound, a precursor or derivative thereof, or a salt of any of the foregoing comprises between 0.01 wt % in the final cosmetic formulation. In certain embodiments of the methods of the present application, the one or more phenoxazone and/or phenoxazine compound, a precursor or derivative thereof, or a salt of any of the foregoing comprises between 0.03 wt % in the final cosmetic formulation.

In certain embodiments of the methods of the present application, the UV-filtering material comprises a UV-absorbing compound. In certain such embodiments, the UV-absorbing compound is one of avobenzone, oxybenzone, oxybenzone cinoxate, homosalate, octisalate, octinoxate, octocrylene, and/or trolamine salicylate. In certain embodiments, the UV-filtering material comprises a UV-scattering particle. In certain such embodiments, the UV-scattering particle comprises titanium dioxide or zinc oxide.

The present application further provides an antioxidant composition comprising a vitamin E analogue; and an unsaturated solution of one or more phenoxazone and/or phenoxazine compound, a precursor or derivative thereof, or a salt of any of the foregoing. In certain such embodiments, the one or more phenoxazone and/or phenoxazine compound, a precursor or derivative thereof, or a salt of any of the foregoing comprises xanthommatin, decarboxylated xanthommatin, uncyclized xanthommatin, ommatin D, dihydroxy-xanthommatin, rhodommatin, or a precursor or derivative thereof, or a salt of any of the foregoing, such as xanthommatin, or a salt thereof. In certain embodiments, the composition performs as an antioxidant for longer than a composition comprising the vitamin E analogue alone. In certain embodiments, the vitamin E analogue and one or more phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin) are present in the composition in a molar ratio of about 1:1. In certain embodiments, the one or more phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin) of the unsaturated solution are present in an amount between about 0.01 and 10 wt %, in particular between 0.01-1 wt %, between 1-5 wt %, between 1-10 wt %, or any other suitable amount. In certain embodiments, the one or more phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin) of the unsaturated solution are present in an amount of about 50 wt %. In certain embodiments, the one or more phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin) of the unsaturated solution are present in an amount of about 50 wt % or up to 75 wt %. In certain embodiments, the vitamin E analogue is Trolox.

The present application provides an antioxidant composition comprising ascorbic acid and an unsaturated solution of one or more phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin). In certain embodiments, the composition performs as an antioxidant for longer than a composition comprising the ascorbic acid alone. In certain embodiments, the one or more phenoxazone and/or phenoxazine compound, a precursor or derivative thereof, or a salt of any of the foregoing comprises xanthommatin, decarboxylated xanthommatin, uncyclized xanthommatin, ommatin D, dihydroxy-xanthommatin, rhodommatin, or a precursor or derivative thereof, or a salt of any of the foregoing, such as xanthommatin, or a salt thereof. In certain embodiments, the ascorbic acid and one or more phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin) of the unsaturated solution are present in a ratio of about 1:1. In certain embodiments, the one or more phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin) of the unsaturated solution are present in the final formulation in an amount between about 0.01 and 10 wt %, in particular between about 0.01-1 wt %, between about 1-5 wt %, between about 1-10 wt %, or any other suitable amount. In certain embodiments, the one or more phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin) of the unsaturated solution are present in an amount of about 50 wt % in the final formulation.

By integrating the unsaturated solution of one or more phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin), into dermatological formulations, and in particular into sun protecting formulations such as sunscreen and facial moisturizer, enhanced protection can be achieved without the systemic toxicities associated with traditional chemical and physical UV filters. Compositions including unsaturated solutions of one or more phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin) provide safer and more effective broad spectrum protection in suncare product formulations, and prolong the efficacy of the formulation.

### BRIEF DESCRIPTION OF THE DRAWINGS

FIG. 1 depicts the antioxidant properties of various concentrations of Trolox compared to those for 1:1 combinations of trolox:xanthommatin.

FIG. 2 depicts the antioxidant properties of various concentrations of ascorbic acid compared to those for 1:1 combinations of ascorbic acid:xanthommatin.

FIG. 3 depicts the antioxidant properties of various concentrations of xanthommatin.

FIG. 4A depicts the UV absorption of combinations of xanthommatin with oxybenzone as compared to oxybenzone alone on day 1. FIG. 4B depicts the UV absorption of combinations of xanthommatin with oxybenzone as compared to oxybenzone alone on day 16.

FIG. 5A depicts the UV absorption of combinations of xanthommatin with avobenzone as compared to avobenzone alone on day 1. FIG. 5B depicts the UV absorption of combinations of xanthommatin with avobenzone as compared to avobenzone alone on day 16.

FIG. 6A depicts the UV absorption of combinations of xanthommatin with octisalate and homosalate (top two lines) as compared to octisalate and homosalate alone on day 1.

FIG. 6B depicts the UV absorption of combinations of xanthommatin with octisalate and homosalate (top two lines) as compared to octisalate and homosalate alone on day 16.

FIG. 7A depicts the UV absorption of combinations of xanthommatin with octinoxate as compared to octinoxate alone on day 1. FIG. 7B depicts the UV absorption of combinations of xanthommatin with octinoxate as compared to octinoxate alone on day 16.

FIG. 8A depicts the UV absorption of combinations of xanthommatin with octocrylene as compared to octocrylene alone on day 1. FIG. 8B depicts the UV absorption of combinations of xanthommatin with octocrylene as compared to octocrylene alone on day 16.

FIG. 9 depicts the UV absorption of octinoxate alone and in combination with avobenzone or xanthommatin.

FIG. 10 depicts the UV absorption of avobenzone alone and in combination with xanthommatin.

FIG. 11 depicts the UV absorption of oxybenzone alone and in combination with avobenzone or xanthommatin.

FIG. 12 depicts results from the measurement of the SPF on chemical sunscreen samples pre-irradiation (solid bars) and post-irradiation (patterned bars).

FIG. 13 depicts results from the measurement of the SPF on mineral sunscreen samples pre-irradiation (solid bars) and post-irradiation (patterned bars).

FIG. 14 depicts the absorbance capabilities of ammonium xanthommatin.

FIG. 15 depicts the absorbance capabilities of ammonium xanthommatin in combination with various FDA approved organic UV filters.

FIG. 16 depicts the absorbance capabilities of ammonium xanthommatin in combination with various FDA approved organic UV filters.

FIG. 17 depicts in vitro phototoxicity measurements of xanthommatin with and without exposure to UVA light. FIG. 17A shows the cell viability for various concentrations of xanthommatin. FIG. 17B shows the cell viability for various concentrations of positive control, chlorpromazine.

FIG. 18 depicts area under the curve (AUC) calculations for the highest concentration (40 uM) conditions for vitamin E, vitamin C, and ammonium xanthommatin (Xa).

FIG. 19 depicts half life measurements of ammonium xanthommatin in solution compared to vitamin C in solution when assayed at the same concentration.

FIG. 20 depicts the activity of vitamin C as measured alone or in combination with ammonium xanthommatin (Xa).

FIG. 21 depicts the activity of vitamin E as measured alone or in combination with ammonium xanthommatin (Xa).

### DETAILED DESCRIPTION

Xanthommatin is biochrome present in arthropods and cephalopods which can be isolated or synthesized and used in formulations as an antioxidant that stabilizes and in some cases boosts or enhances the UV-filtering performance of chemical sunscreen agents. The bioinspired molecule is a safe and cytocompatible alternative to traditional chemicals used in suncare products for enhancing and boosting existing UV-filters and anti-oxidizing compounds or as a functional alternative UVA filter. The compositions disclosed herein use unsaturated solutions of one or more phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin) as an active ingredient in dermatological protection compositions for sun protection or other dermatological protection, and provides a safer and more effective alternative to chemicals traditionally utilized in such compositions that can have harmful health consequences.

Formulating phenoxazone and/or phenoxazine compounds, precursors or derivatives thereof, or salts of any of the foregoing (for example xanthommatin, decarboxylated xanthommatin, uncyclized xanthommatin, ommatin D, dihydroxy-xanthommatin, or rhodommatin, precursors or derivatives thereof, or salts of any of the foregoing) as a standalone, unaggregated unit in an unsaturated solution has a number of surprising advantages. For example, it can boost the performance of UV filters. As described in the exemplification section with reference to the accompanying Figures, preliminary data supports findings that unsaturated solutions of xanthommatin, or a salt thereof, can increase the photo-stability or otherwise stabilize the UV-absorptive properties of known chemical UV-filters. Unsaturated solutions of xanthommatin, or a salt thereof, are capable of replacing entirely or competing with the performance of the only other known UV-A filter, avobenzone. Unsaturated solutions of xanthommatin, or a salt thereof, can provide improved performance compared to known antioxidants in terms of antioxidant properties and stability over time.

The compositions disclosed herein include unsaturated solutions of one or more phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin) as a soluble portion or solution phase of an emulsion (such as a cream) or homogenously distributed within a suspension (such as an aerosol spray).

Each of these functions of unsaturated solutions of one or more phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin) in a non-aggregated and non-particle form, are discussed briefly in turn.

General Use

The present application provides a composition comprising an unsaturated solution of 1 wt % or less of one or more phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin) and at least one UV-filtering material (e.g., a UV-absorbing compound or a UV-scattering particle). In certain embodiments, the composition is for use in providing broad spectrum protection. In certain embodiments, the phenoxazone and/or phenoxazine compound comprises, for example, xanthommatin, decarboxylated xanthommatin, uncyclized xanthommatin, ommatin D, dihydroxy-xanthommatin, rhodommatin, a derivative or precursor thereof, or a salt of any of the foregoing. In certain embodiments, the phenoxazone and/or phenoxazine compound, a precursor or derivative thereof, or a salt of any of the foregoing is present in the composition in an amount less than about 1.0 wt %, 0.9 wt %, 0.8 wt %, 0.7 wt, 0.6 wt %, 0.5 wt %, 0.4 wt %, 0.3 wt %, 0.1 wt %, 0.05 wt %, or 0.01 wt %. In certain embodiments, the phenoxazone and/or phenoxazine compound, a precursor or derivative thereof, or a salt of any of the foregoing is present in the composition in a range of about wt %0.1-1 wt %, 0.01-0.1 wt %, 0.01-0.05 wt %, or any other suitable amount. In certain embodiments, the UV-filtering material (e.g., a UV-absorbing compound or a UV-scattering particle) is present in the composition in an amount greater than about 0.1 wt %, 1 wt %, 5 wt %, 10 wt %, 15 wt %, 20 wt %, 25 wt %, 30 wt %, 35 wt %, or 40 wt % or other suitable amount. In some embodiments, the UV-filtering material (e.g., a UV-absorbing compound or a UV-scattering particle) is present in the final formulation in a range of about 0.1-40 wt %, 0.1-35 wt %, 0.1-30 wt %, 0.1-25 wt %, 0.1-20 wt %, 0.1-15 wt %, 0.1-5 wt %, or 0.1-1 wt %.

The present application provides a composition comprising an unsaturated solution of 0.1-1 wt % of one or more phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin), and 0.1-40 wt % of a UV-filtering material (e.g., a UV-absorbing compound or a UV-scattering particle). In certain embodiments, the composition has an SPF of about 30-100 and UVA-PF of about 30-100.

In certain embodiments of the compositions of the application, the at least one UV-filtering material (e.g., a UV-absorbing compound or a UV-scattering particle) and the unsaturated solution of 1 wt % or less of one or more phenoxazone and/or phenoxazine compound, a precursor or derivative thereof, or a salt of any of the foregoing together exhibit synergy. When the efficacy (e.g., the SPF or the UV absorbing properties) of the mixture of the two or more ingredients exceeds the efficacy of each ingredient, it is called “synergy.” In certain embodiments, the at least one UV-filtering material (e.g., a UV-absorbing compound or a UV-scattering particle) and the unsaturated solution of 1 wt % or less of one or more phenoxazone and/or phenoxazine compound, a precursor or derivative thereof, or a salt of any of the foregoing together provide more than the anticipated efficacy (e.g., the SPF or the UV-absorbing and/or -scattering properties) of combining the at least one UV-filtering material (e.g., a UV-absorbing compound or a UV-scattering particle) and the unsaturated solution of 1 wt % or less of one or more phenoxazone and/or phenoxazine compound, a precursor or derivative thereof, or a salt of any of the foregoing. In certain such embodiments, the combination provides at least 5%, 10%, 15%, 20%, 25%, or 30% more than the anticipated efficacy (e.g., the SPF or the UV-absorbing and/or -scattering properties).

In certain embodiments, the composition has a pre-irradiation SPF greater than a composition comprising the at least one UV-filtering material (e.g., a UV-absorbing compound or a UV-scattering particle) without the unsaturated solution of 1 wt % or less of one or more phenoxazone and/or phenoxazine compound, a precursor or derivative thereof, or a salt of any of the foregoing. In certain such embodiments, the composition has an increase in pre-irradiation SPF of greater than 1%, greater than 5%, greater than 10%, greater than 15%, greater than 20%, greater than 25%, greater than 30%, greater than 35%, greater than 40%, greater than 45%, or greater than 50% as compared to a composition comprising the at least one UV-filtering material (e.g., a UV-absorbing compound or a UV-scattering particle) without the unsaturated solution of 1 wt % or less of one or more phenoxazone and/or phenoxazine compound, a precursor or derivative thereof, or a salt of any of the foregoing. In certain embodiments, the composition has a post-irradiation SPF greater than a composition comprising the at least one UV-filtering material (e.g., a UV-absorbing compound or a UV-scattering particle) without the unsaturated solution of 1 wt % or less of one or more phenoxazone and/or phenoxazine compound, a precursor or derivative thereof, or a salt of any of the foregoing. In certain such embodiments, the composition has an increase in post-irradiation SPF of greater than 1%, greater than 5%, greater than 10%, greater than 15%, greater than 20%, greater than 25%, greater than 30%, greater than 35%, greater than 40%, greater than 45%, or greater than 50% as compared to a composition comprising the at least one UV-filtering material (e.g., a UV-absorbing compound or a UV-scattering particle) without the unsaturated solution of 1 wt % or less of one or more phenoxazone and/or phenoxazine compound, a precursor or derivative thereof, or a salt of any of the foregoing. In certain embodiments, the composition has a pre- and post-irradiation SPF greater than a composition comprising the at least one UV-filtering material (e.g., a UV-absorbing compound or a UV-scattering particle) without the unsaturated solution of 1 wt % or less of one or more phenoxazone and/or phenoxazine compound, a precursor or derivative thereof, or a salt of any of the foregoing.

In certain embodiments, the SPF of the composition is maintained for at least 1 day, at least 2 days, at least 3 days, at least 4 days, at least 5 days, at least 6 days, at least 1 week, at least 2 weeks, at least 3 weeks, at least 4 weeks, or at least 5 weeks. In certain embodiments, the SPF of the composition is maintained for at least 1 week. In certain embodiments, the SPF of the composition is maintained for at least 2 weeks. In certain embodiments, the SPF of the composition is maintained for at least 3 weeks. In certain embodiments of the foregoing, the SPF is pre-irradiation SPF.

In certain embodiments, the composition exhibits greater UV absorbance than a composition comprising the at least one UV-filtering material (e.g., a UV-absorbing compound or a UV-scattering particle) without the unsaturated solution of 1 wt % or less of one or more phenoxazone and/or phenoxazine compound, a precursor or derivative thereof, or a salt of any of the foregoing. In certain such embodiments, the composition exhibits a 10%, 20%, 30%, 40%, 50%, 60%, 70%, 80%, 90%, 100%, 125%, 150%, 175%, 200%, 225%, or 250% increase in UV absorbance as compared to a composition comprising the at least one UV-filtering material (e.g., a UV-absorbing compound or a UV-scattering particle) without the unsaturated solution of 1 wt % or less of one or more phenoxazone and/or phenoxazine compound, a precursor or derivative thereof, or a salt of any of the foregoing.

In certain embodiments, the one or more UV-filtering material comprises one or more UV-absorbing compound, such as one or more chemical sunblock. In certain embodiments, the one or more UV-filtering material comprises one or more UV-scattering compound, such as one or more physical sunblock. In certain embodiments, the one or more UV-absorbing compound is selected from avobenzone, oxybenzone, oxybenzone cinoxate, homosalate, octisalate, octinoxate, octocrylene, and trolamine salicylate, bemotrizinol, and bisoctrizole. In certain embodiments, the UV-filtering material comprises chemical and physical sunblocks. Non-limiting examples of chemical sunblocks that can be used include bemotrizinol (Tinosorb S), bisoctrizole (Tinosorb M), para-aminobenzoic acid (PABA), PABA esters (glyceryl PABA, amyldimethyl PABA and octyldimethyl PABA), butyl PABA, ethyl PABA, ecamsule, ethyl dihydroxypropyl PABA, benzophenones (oxybenzone, sulisobenzone, benzophenone, and benzophenone-1 through 12), cinnamates (octyl methoxycinnamate, isoamyl p-methoxycinnamate, octylmethoxy cinnamate, cinoxate, diisopropyl methyl cinnamate, DEA-methoxycinnamate, ethyl diisopropylcinnamate, glyceryl octanoate dimethoxycinnamate and ethyl methoxycinnamate), cinnamate esters, salicylates (homomethyl salicylate, benzyl salicylate, glycol salicylate, isopropylbenzyl salicylate, etc.), anthranilates, ethyl urocanate, homosalate, octisalate, dibenzoylmethane derivatives (e.g., avobenzone), octocrylene, octyl triazone, digalloy trioleate, glyceryl aminobenzoate, lawsone with dihydroxyacetone, ethylhexyl triazone, dioctyl butamido triazone, benzylidene malonate polysiloxane, terephthalylidene dicamphor sulfonic acid, disodium phenyl dibenzimidazole tetrasulfonate, diethylamino hydroxybenzoyl hexyl benzoate, bis diethylamino hydroxybenzoyl benzoate, bis benzoxazoylphenyl ethylhexylimino triazine, drometrizole trisiloxane, methylene bis-benzotriazolyl tetramethylbutylphenol, and bis-ethylhexyloxyphenol methoxyphenyltriazine, 4-methylbenzylidenecamphor, and isopentyl 4-methoxycinnamate. Non-limiting examples of physical sunblocks include kaolin, talc, petrolatum and metal oxides (e.g., titanium dioxide and zinc oxide). In certain embodiments, the UV-filtering material (e.g., a UV-absorbing compound or a UV-scattering particle) is present in the composition at more than 1 wt %, more than 2 wt %, more than 3 wt %, more than 4 wt %, more than 5 wt %, more than 10 wt %, more than 15 wt %, more than 20 wt %, more than 25 wt %, more than 30 wt %, more than 35 wt %, more than 40 wt %, more than 45 wt %, or more than 50 wt %.

The compositions of the present application can be formulated as a solution, suspension or emulsion. In certain embodiments, the one or more phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin) is distributed with the UV-filtering material (e.g., a UV-absorbing compound or a UV-scattering particle) within the solution, suspension or emulsion. In certain such embodiments, the distribution may be homogeneous. In certain embodiments, the compositions of the present application are formulated as a cream, gel, spray, or lotion. In certain such embodiments, the compositions are for use in a cosmetic or dermatological formulation. The compositions of the present application are suitable for topical use to provide protection from solar ultraviolet radiation.

The present application provides a method of providing protection from solar ultraviolet radiation, comprising applying a cosmetic or dermatological formulation comprising an unsaturated solution of 1 wt % or less one or more phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin), and 0.1-40 wt % of a UV-filtering material (e.g., a UV-absorbing compound or a UV-scattering particle). In certain embodiments, the composition comprises 0.1-1 wt % of the unsaturated solution of one or more phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin). In certain embodiments, the composition comprises 10-40 wt % of the UV-filtering material (e.g., a UV-absorbing compound or a UV-scattering particle). In certain embodiments, the cosmetic or dermatological formulation is for topical administration.

In certain embodiments, the compositions of the present application further comprise non-ionic polymeric emulsifiers such as potassium cetyl phosphate, PEG-150 distearate, cetearyl alcohol, caprylic/capric triglyceride, and glyceryl stearate.

In certain embodiments, the compositions of the present application provide sun protection. In certain embodiments, the compositions provide an SPF of at least about 15, 30, 60, 100, or other suitable amount of protection. In certain embodiments, the amount of phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin) in the composition may be altered or adjusted in order to provide a particular SPF. In certain embodiments, the amount of UV-filtering material (e.g., a UV-absorbing compound or a UV-scattering particle) in the composition may be altered or adjusted in order to provide a particular SPF. For example, xanthommatin has a critical wavelength of 385 nm, while FDA-approved UV-filters are considered “broad-spectrum” if they have a critical wavelength of at least 370 nm. Accordingly, xanthommatin in an unsaturated solution can function as a broad spectrum UV-filter. The SPF provided by xanthommatin formulations varies with concentration, such that xanthommatin is a tunable UV-filter. A formulation of 0.03 mM xanthommatin in a solution provides about 1 SPF, 0.25 mM concentration provides about 5 SPF, 1 mM concentration provides about 20 SPF, and 5 mM concentration is anticipated to provide about 100 SPF. In certain embodiments, the composition is formulated to provide an UVA-PF of at least 15. For example, the composition can be formulated to provide UVA-PF of at least 15, 30, 60, 100, or any other suitable amount of protection. The amounts of phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin) and/or UV-filtering material (e.g., a UV-absorbing compound or a UV-scattering particle) in the composition may be altered or adjusted in order to provide a particular SPF. In certain embodiments, the composition provides broad spectrum protection. In certain embodiments, the composition provides a ratio of UVA I to UV filters of at least 0.7 or greater.

In certain embodiments of the compositions of the present application, the unsaturated solution of phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin), is relied on for its anti-oxidizing properties. The amount of phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin) present in the composition may be increased or otherwise adjusted in order to achieve the required anti-oxidizing performance. In certain embodiments, the compositions of the present application may comprise a further anti-oxidizing compound. In certain such embodiments, the further anti-oxidizing compound comprises one or more of arbutin, BHA, BHT, koji acid, hydroxyanisole, hydroquinone, t-butyl hydroquinone, tocopherol, nordihydroguaiaretic acid, rosmarinic acid, Trolox, goosypol, flavone, flavanone, isoflavones, flavanol, protocatechuic acid, resorcylic acid, gallic, caffeic acid, ferulic acid, chlorogenic acid, ascorbic acid, ascorbyl palmitate, carotenoids, cysteine hydrochloride, dithiothreitol, glutathione, thioglycolic acid, thiodipropionic acid, alpha-lipoic acid, and/or xanthines. In certain embodiments, the further anti-oxidizing compound comprises include acetyl cysteine, alpha-lipoic acid, arbutin, ascorbic acid, ascorbic acid polypeptide, ascorbyl dipalmitate, ascorbyl methylsilanol pectinate, ascorbyl palmitate, ascorbyl stearate, BHA, BHT, t-butyl hydroquinone, caffeic acid, carotenoids, chlorogenic acid, cysteine, cysteine HCl, diamyihydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, flavone, flavanone, flavanol, gallic acid esters, glutathione, goosypol, hydroquinone, hydroxyanisole, isoflavones, isooctyl thioglycolate, ithiothreitol, kojic acid, magnesium ascorbate, magnesium ascorbyl phosphate, methylsilanol ascorbate, natural botanical antioxidants such as green tea or grape seed extracts, nordihydroguaiaretic acid, octyl gallate, phenylthioglycolic acid, potassium ascorbyl tocopheryl phosphate, potassium sulfite, propyl gallate, protocatechuic acid, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thiodipropionic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, tris(nonylphenyl)phosphite, Trolox, and xanthines. In certain embodiments, the further anti-oxidizing compound is present in the composition in an amount greater than 0.1 wt %, 0.5 wt %, 1 wt %, 3 wt %, 5 wt %, or any other suitable amount.

In certain embodiments, the compositions of the present application further comprise an anti-radical compound. In certain such embodiments, the anti-radical compound is present in the final formulation in an amount greater than 0.1 wt %, 1 wt %, 5 wt %, 6 wt %, 8 wt %, 10 wt %, or any other suitable amount.

The present application further provides a composition comprising an unsaturated solution of one or more phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin) and a UV-filtering or anti-oxidizing component. In certain embodiments, the unsaturated solution of one or more phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin) enhances the performance of the UV-filtering or anti-oxidizing component. By homogeneously distributing the unsaturated solution of phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin), in the composition the active UV-filtering or anti-oxidizing component, the unsaturated solution is able to improve the UV-filtering or ant-oxidizing performance of the UV-filtering or anti-oxidizing component.

UV-Filter Stabilizer

An unsaturated solution of 1 wt % or less of one or more phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin), may act as a stand-alone unaffiliated stabilizer when distributed in a composition with an active UV-filtering material (e.g., a UV-absorbing compound or a UV-scattering particle). Accordingly, the present application provides a composition comprising an unsaturated solution of 1 wt % or less of one or more phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin), and a UV-filtering material (e.g., a UV-absorbing compound or a UV-scattering particle), wherein the one or more phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin) stabilizes the UV-filtering material (e.g., a UV-absorbing compound or a UV-scattering particle). In certain embodiments, greater than 10%, 20%, 30%, 40%, 50%, 60%, 70%, 80%, or 90% of the initial UV-absorbing capacity is retained for the UV-absorbing compound for at least one week, at least two weeks, or at least three weeks. In certain embodiments, 100% of the initial UV-absorbing capacity is retained for the UV-absorbing compound for at least one week, at least two weeks, or at least three weeks. In certain embodiments, greater than 10%, 20%, 30%, 40%, 50%, 60%, 70%, 80%, or 90% of the initial UV-scattering capacity is retained for the UV-scattering particle for at least one week, at least two weeks, or at least three weeks. In certain embodiments, 100% of the initial UV-scattering capacity is retained for the UV-scattering particle for at least one week, at least two weeks, or at least three weeks.

The present application further provides a method of stabilizing a UV-filtering material (e.g., a UV-absorbing compound or a UV-scattering particle), comprising combining the UV-filtering material (e.g., a UV-absorbing compound or a UV-scattering particle) with an unsaturated solution of 1 wt % or less of one or more phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin). In certain embodiments, the stabilizing the UV-filtering material (e.g., a UV-absorbing compound or a UV-scattering particle) comprises retaining greater than 10%, 20%, 30%, 40%, 50%, 60%, 70%, 80%, or 90% of the UV-absorbing and/or UV-scattering capacity for at least one week, at least two weeks, or at least three weeks.

In certain embodiments, the UV-filtering material (e.g., a UV-absorbing compound or a UV-scattering particle) comprises a photo-unstable chemical UV-filter, for example avobenzone, oxybenzone, oxybenzone cinoxate, homosalate, octisalate, octinoxate, octocrylene, and/or trolamine salicylate. These UV-light absorbing chemical filters are typically unstable and require stabilization by one or more compounds or materials in the formulation.

In certain embodiments, the phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin) is present in an amount between about 0.01-0.05 wt %, 0.1-1 wt %, of the composition.

As described below with regard to the Examples and accompanying Figures, the presence of unaggregated xanthommatin was shown to contribute to a boost in UVB and UVA stability of commercially available UV-filters, when exposed to ambient light and temperatures. This boost in stability in the presence of unsaturated solutions of xanthommatin was consistent across all chemical filters tested, including homosalate, octisalate, octocrylene, oxybenzone, and octinoxate. Between 10-100% UV filtering retention was observed over the course of weeks. Solutions including unsaturated solutions of one or more phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin) may also boost the stability of UV-filters in the presence of solar simulated light, or solar light.

UV-Absorbance Booster

An unsaturated solution of 1 wt % or less of one or more phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin), can be used as a UV-absorbance booster for known UV-absorbing compounds. Accordingly, the present application provides a composition comprising an unsaturated solution of 1 wt % or less of one or more phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin), and an active UV-absorbing compound, wherein the one or more phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin) enhances the UV-absorbing properties of the UV-absorbing compound. In certain embodiments, the composition enhances the UV-absorbing properties of the UV-absorbing compound in UV-B. In certain embodiments, the composition enhances the UV-absorbing properties of the UV-absorbing compound in UV-A. In certain embodiments, the composition enhances the UV-absorbing properties of the UV-absorbing compound in UV-A and UV-B.

The present application further provides a method of enhancing the UV-absorbing properties of a UV-absorbing compound, comprising combining the UV-absorbing compound with an unsaturated solution of 1 wt % or less of one or more phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin).

In certain embodiments, the one or more phenoxazone and/or phenoxazine compound, a precursor or derivative thereof, or a salt of any of the foregoing, comprises xanthommatin, decarboxylated xanthommatin, uncyclized xanthommatin, ommatin D, dihydroxy-xanthommatin, rhodommatin, a precursor or derivative thereof, or a salt of any of the foregoing. In certain embodiments, the one or more phenoxazone and/or phenoxazine compound, a precursor or derivative thereof, or a salt of any of the foregoing, comprises 0.01-0.03 wt %, 0.1-1 wt %, or 0.1-1 wt %, of the composition. In certain such embodiments, the one or more phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin), comprises 0.03 wt % of the composition.

In certain embodiments, the UV-absorbing compound comprises avobenzone, oxybenzone, homosalate, octisalate, octinoxate, octocrylene, oxybenzone cinoxate, and/or trolamine salicylate. In certain embodiments, the UV-absorbing compound comprises titanium dioxide or zinc oxide.

The simple addition of an unsaturated solution of xanthommatin at concentrations ranging from 0.01-0.03 wt %, 0.1-1 wt %, 0.1-1 wt %, or 1-10 wt % can induce significant spectral enhancements in the UV-absorption properties of commercially available chemical UV-filters. In one example, an unsaturated solution of xanthommatin incorporated into a composition at about 0.03 wt % xanthommatin can boost activity of avobenzone by ˜250% in UV-B (300 nm) and about 9% in UV-A (360 nm). This differs from activity of avobenzone with oxybenzone or avobenzone with octinoxate which only boost UV-B performance of avobenzone by 229% and 393%, respectively. (See Examples and accompanying Figures).

When looking at UV-A performance oxybenzone and octinoxate reduce avobenzone performance by about 46% and 56%, respectively. These major differences highlight the important capabilities of xanthommatin as a booster.

Alternative UV-A Filter

Unsaturated solutions of one or more phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin), can perform as an alternative UV-A filter to the photo-unstable avobenzone, which is currently the only US-approved chemical UV-A filter.

As described below with regard to the Examples and accompanying Figures, in experiments the use of unaggregated xanthommatin with oxybenzone had absorption intensities in solution of about 1.19 (compared to avobenzone and oxybenzone at 0.79 intensity measured at 360 nm). This trend was similarly observed with unaggregated xanthommatin with octinoxate where intensities of about 1.09 in solution were achieved, compared to octinoxate and avobenzone which resulted in an intensity of about 0.65 at 360 nm.

Antioxidant Booster and Stabilizer

An unsaturated solution of one or more phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin), can perform as an antioxidant booster. Accordingly, the present application provides a composition comprising an unsaturated solution of one or more phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin), and an antioxidant, wherein the one or more phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin) enhances antioxidant capacity of the antioxidant. In certain embodiments, the antioxidant comprises the vitamin E analogue Trolox or ascorbic acid.

The present application provides a method of enhancing the antioxidant capacity of an antioxidant, comprising combining the antioxidant with an unsaturated solution of one or more phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin).

An unsaturated solution of one or more phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin) can perform as a direct replacement for existing and known antioxidants in cosmetic applications. Accordingly, the present application provides an unsaturated solution of one or more phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin) for use in a cosmetic composition, wherein the unsaturated solution provides antioxidant properties for the cosmetic composition.

An unsaturated solution of one or more phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin), can perform as a stabilizer to the same antioxidants. Accordingly, the present application provides a composition comprising one or more phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin), and an antioxidant, wherein the one or more phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin) stabilizes the antioxidant. In certain embodiments, the one or more phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin) and the antioxidant are present in the composition in a 0.1:1, 0.2:1, 0.3:1, 0.4:1, 0.5:1, 0.6:1, 0.7:1, 0.8:1, 0.9:1, or 1:1 molar ratio. In certain embodiments, the one or more phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin) and the antioxidant are present in the composition in a 2:1, 3:1, 4:1, 5:1, 6:1, 7:1, 8:1, 9:1, or 10:1 molar ratio. In certain embodiments, the one or more phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin) and the antioxidant are present in the composition in a 1:1 molar ratio. In certain embodiments, the antioxidant comprises the vitamin E analogue Trolox or ascorbic acid. For example, a composition of about 1:1 vitamin E analogue such as Trolox and xanthommatin shows evidence heightened antioxidant stability. Similarly, a composition of 1:1 ascorbic acid and xanthommatin also shows antioxidant stabilization.

The present application provides a method of maintaining the antioxidant capacity of an antioxidant, comprising combining the antioxidant with an unsaturated solution of one or more phenoxazone and/or phenoxazine compound, a precursor or derivative thereof, or a salt of any of the foregoing (e.g., xanthommatin, a precursor or derivative thereof, or a salt of any one of the foregoing, such as ammonium xanthommatin). In certain embodiments, 100% of the antioxidant capacity of an antioxidant is maintained for at least one week. In certain embodiments, at least 30%, at least 40%, at least 50%, at least 60%, at least 70%, at least 80%, or at least 90% of the antioxidant capacity of an antioxidant is maintained for at least one week.

In certain embodiments of the foregoing methods and compositions, the one or more phenoxazone and/or phenoxazine compound, a precursor or derivative thereof, or a salt of any of the foregoing, comprises xanthommatin, decarboxylated xanthommatin, uncyclized xanthommatin, ommatin D, dihydroxy-xanthommatin, rhodommatin, a precursor or derivative thereof, or a salt of any of the foregoing. In certain embodiments, the one or more phenoxazone and/or phenoxazine compound, a precursor or derivative thereof, or a salt of any of the foregoing, comprises 0.01-1 wt %, 1-5 wt %, 1-10 wt %, 50 wt %, or up to 75% of the composition. wt % wt % wt % wt %.

As described below and in the accompanying Figures, antioxidant stabilizing/boosting effects were observed in experiments in which the anti-oxidizing capacity and performance of unsaturated solutions of xanthommatin only and xanthommatin were blended with equimolar ascorbic acid (vitamin C) or Trolox (a vitamin E analogue) over 90 minutes at 37° C. using a standard Oxygen Radical Antioxidant Capacity (ORAC) assay. The ORAC assay measures the antioxidant capacity of biomolecules based on the oxidation of a fluorescein probe by peroxyl radicals (Fluorescence decreases as antioxidant capacity decreases). In all cases, xanthommatin in an unsaturated solution provided comparable anti-oxidizing capacity to known antioxidant standards (Trolox and vitamin C). These findings are supported by the comparable area under the curve (AUC) calculations for the highest concentration (40 μM) conditions.

Cosmetic or Dermatological Formulation

The present application provides cosmetic or dermatological formulations comprising the compositions as disclosed herein.

In certain embodiments, the cosmetic or dermatological formulations comprising the compositions as disclosed herein further comprise a rheology modifier. In certain embodiments, the rheology modifier is present in an amount that prevents significant dripping or pooling of the composition after application to the skin. In certain embodiments, the rheology modifier is carbomer. In some embodiments, the rheology modifier is selected from stearic acid, palmitic acid, stearyl alcohol, cetyl alcohol, behenyl alcohol, stearic acid, palmitic acid, the polyethylene glycol ether of stearyl alcohol having an average of about 1 to about 21 ethylene oxide units, the polyethylene glycol ether of cetyl alcohol having an average of about 1 to about 5 ethylene oxide units, and mixtures thereof.

Additional examples of rheology modifiers include thickener or gelling agents, including substances which that can increase the viscosity of a composition. Thickening agents include those that can increase the viscosity of a composition without substantially modifying the efficacy of the active ingredient within the composition. Thickening agents can also increase the stability of the compositions of the present application. In certain aspects of the present application, thickening agents include hydrogenated polyisobutene or trihydroxy stearin, or a mixture of both. Additional non-limiting examples of additional thickening agents that can be used in the context of the present application include carboxylic acid polymers, crosslinked polyacrylate polymers, polyacrylamide polymers, polysaccharides, and gums. Examples of carboxylic acid polymers include crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol (see CTFA International Cosmetic Ingredient Dictionary, Fourth Edition, 1991, pp. 12 and 80). Examples of commercially available carboxylic acid polymers include carbomers, which are homopolymers of acrylic acid crosslinked with allyl ethers of sucrose or pentaerythritol (e.g., Carbopol™ 900 series from B. F. Goodrich). Non-limiting examples of crosslinked polyacrylate polymers include cationic and nonionic polymers.

Non-limiting examples of polyacrylamide polymers (including nonionic polyacrylamide polymers including substituted branched or unbranched polymers) include polyacrylamide, isoparaffin and Laureth-7, multi-block copolymers of acrylamides and substituted acrylamides with acrylic acids and substituted acrylic acids.

Non-limiting examples of polysaccharides include cellulose, carboxymethyl hydroxyethylcellulose, cellulose acetate propionate carboxylate, hydroxyethylcellulose, hydroxyethyl ethylcellulose, hydroxypropyl cellulose, hydroxypropyl methylcellulose, methyl hydroxyethylcellulose, microcrystalline cellulose, sodium cellulose sulfate, and mixtures thereof. Another example is an alkyl substituted cellulose where the hydroxy groups of the cellulose polymer are hydroxyalkylated (preferably hydroxy ethylated or hydroxypropylated) to form a hydroxyalkylated cellulose which is then further modified with a C10-C30 straight chain or branched chain alkyl group through an ether linkage. Typically these polymers are ethers of C10-C30 straight or branched chain alcohols with hydroxyalkylcelluloses. Other useful polysaccharides include scleroglucans comprising a linear chain of (1-3) linked glucose units with a (1-6) linked glucose every three unit.

Non-limiting examples of gums that can be used with the present compositions include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboyxmethyl dextran, sodium carrageenan, tragacanth gum, xanthan gum, and mixtures thereof. In one embodiment, the thickening agent is Chondrus crispus (carrageenan) extract.

In certain embodiments, the cosmetic or dermatological formulations comprising the compositions as disclosed herein further comprise a moisturizing agent (e.g., humectant). Examples of moisturizing agents that can be used in the cosmetic or dermatological formulations of the present application include amino acids, chondroitin sulfate, diglycerin, erythritol, fructose, glucose, glycerin, glycerol polymers, glycol, 1,2,6-hexanetriol, honey, hyaluronic acid, hydrogenated honey, hydrogenated starch hydrolysate, inositol, lactitol, maltitol, maltose, mannitol, natural moisturizing factor, PEG-15 butanediol, polyglyceryl sorbitol, salts of pyrollidone carboxylic acid, potassium PCA, propylene glycol, sodium glucuronate, sodium PCA, sorbitol, sucrose, trehalose, urea, and xylitol. In one embodiment, the moisturizing agent is glycerin. Other examples include acetylated lanolin, acetylated lanolin alcohol, alanine, algae extract, Aloe barbadensis, Aloe-barbadensis extract, Aloe barbadensis gel, Althea officinalis extract, apricot (prunus armeniaca) kernel oil, arginine, arginine aspartate, Arnica montana extract, aspartic acid, avocado (Persea gratissima) oil, barrier sphingolipids, butyl alcohol, beeswax, behenyl alcohol, beta-sitosterol, birch (Betula alba) bark extract, borage (Borago officinalis) extract, butcherbroom (Ruscus aculeatus) extract, butylene glycol, Calendula officinalis extract, Calendula officinalis oil, candelilla (Euphorbia cerifera) wax, canola oil, caprylic/capric triglyceride, cardamon (Elettaria cardamomum) oil, carnauba (Copernicia cerifera) wax, carrot (Daucus carota sativa) oil, castor (Ricinus communis) oil, ceramides, ceresin, ceteareth-5, ceteareth-12, ceteareth-20, cetearyl octanoate, ceteth-20, ceteth-24, cetyl acetate, cetyl octanoate, cetyl palmitate, chamomile (Anthemis nobilis) oil, cholesterol, cholesterol esters, cholesteryl hydroxystearate, citric acid, clary (Salvia sclarea) oil, cocoa (Theobroma cacao) butter, coco-caprylate/caprate, coconut (Cocos nucifera) oil, collagen, collagen amino acids, corn (Zea mays) oil, fatty acids, decyl oleate, dimethicone copolyol, dimethiconol, dioctyl adipate, dioctyl succinate, dipentaerythrityl hexacaprylate/hexacaprate, DNA, erythritol, ethoxydiglycol, ethyl linoleate, Eucalyptus globulus oil, evening primrose (Oenothera biennis) oil, fatty acids, Geranium maculatum oil, glucosamine, glucose glutamate, glutamic acid, glycereth-26, glycerin, glycerol, glyceryl distearate, glyceryl hydroxystearate, glyceryl laurate, glyceryl linoleate, glyceryl myristate, glyceryl oleate, glyceryl stearate, glyceryl stearate SE, glycine, glycol stearate, glycol stearate SE, glycosaminoglycans, grape (Vitis vinifera) seed oil, hazel (Corylus americana) nut oil, hazel (Corylus avellana) nut oil, hexylene glycol, hyaluronic acid, hybrid safflower (Carthamus tinctorius) oil, hydrogenated castor oil, hydrogenated coco-glycerides, hydrogenated coconut oil, hydrogenated lanolin, hydrogenated lecithin, hydrogenated palm glyceride, hydrogenated palm kernel oil, hydrogenated soybean oil, hydrogenated tallow glyceride, hydrogenated vegetable oil, hydrolyzed collagen, hydrolyzed elastin, hydrolyzed glycosaminoglycans, hydrolyzed keratin, hydrolyzed soy protein, hydroxylated lanolin, hydroxyproline, isocetyl stearate, isocetyl stearoyl stearate, isodecyl oleate, isopropyl isostearate, isopropyl lanolate, isopropyl myristate, isopropyl palmitate, isopropyl stearate, isostearamide DEA, isostearic acid, isostearyl lactate, isostearyl neopentanoate, jasmine (Jasminum officinale) oil, jojoba (Buxus chinensis) oil, kelp, kukui (Aleurites moluccana) nut oil, lactamide MEA, laneth-16, laneth-10 acetate, lanolin, lanolin acid, lanolin alcohol, lanolin oil, lanolin wax, lavender (Lavandula angustifolia) oil, lecithin, lemon (Citrus medica limonum) oil, linoleic acid, linolenic acid, Macadamia ternifolia nut oil, maltitol, matricaria (Chamomilla recutita) oil, methyl glucose sesquistearate, methylsilanol PCA, mineral oil, mink oil, mortierella oil, myristyl lactate, myristyl myristate, myristyl propionate, neopentyl glycol dicaprylate/dicaprate, octyldodecanol, octyldodecyl myristate, octyldodecyl stearoyl stearate, octyl hydroxystearate, octyl palmitate, octyl salicylate, octyl stearate, oleic acid, olive (Olea europaea) oil, orange (Citrus aurantium dulcis) oil, palm (Elaeis guineensis) oil, palmitic acid, pantethine, panthenol, panthenyl ethyl ether, paraffin, PCA, peach (Prunus persica) kernel oil, peanut (Arachis hypogaea) oil. Additional non-limiting examples of moisturizing agents may include PEG-8 C12-18 ester, PEG-15 cocamine, PEG-150 distearate, PEG-60 glyceryl isostearate, PEG-5 glyceryl stearate, PEG-30 glyceryl stearate, PEG-7 hydrogenated castor oil, PEG-40 hydrogenated castor oil, PEG-60 hydrogenated castor oil, PEG-20 methyl glucose sesquistearate, PEG40 sorbitan peroleate, PEG-5 soy sterol, PEG-10 soy sterol, PEG-2 stearate, PEG-8 stearate, PEG-20 stearate, PEG-32 stearate, PEG40 stearate, PEG-50 stearate, PEG-100 stearate, PEG-150 stearate, pentadecalactone, peppermint (Mentha piperita) oil, petrolatum, phospholipids, polyamino sugar condensate, polyglyceryl-3 diisostearate, polyquaternium-24, polysorbate 20, polysorbate 40, polysorbate 60, polysorbate 80, polysorbate 85, potassium myristate, potassium palmitate, propylene glycol, propylene glycol dicaprylate/dicaprate, propylene glycol dioctanoate, propylene glycol dipelargonate, propylene glycol laurate, propylene glycol stearate, propylene glycol stearate SE, PVP, pyridoxine dipalmitate, retinol, retinol palmitate, rice (Oryza sativa) bran oil, RNA, rosemary (Rosmarinus officinalis) oil, rose oil, safflower (Carthamus tinctorius) oil, sage (Salvia officinalis) oil, sandalwood (Santalum album) oil, serine, serum protein, sesame (Sesamum indicum) oil, shea butter (Butyrospermum parkii), silk powder, sodium chondroitin sulfate, sodium hyaluronate, sodium lactate, sodium palmitate, sodium PCA, sodium polyglutamate, soluble collagen, sorbitan laurate, sorbitan oleate, sorbitan palmitate, sorbitan sesquioleate, sorbitan stearate, sorbitol, soybean (Glycine soja) oil, sphingolipids, squalane, squalene, stearamide MEA-stearate, stearic acid, stearoxy dimethicone, stearoxytrimethylsilane, stearyl alcohol, stearyl glycyrrhetinate, stearyl heptanoate, stearyl stearate, sunflower (Helianthus annuus) seed oil, sweet almond (Prunus amygdalus dulcis) oil, synthetic beeswax, tocopherol, tocopheryl acetate, tocopheryl linoleate, tribehenin, tridecyl neopentanoate, tridecyl stearate, triethanolamine, tristearin, urea, vegetable oil, water, waxes, wheat (Trificum vulgare) germ oil, and ylang (Cananga odorata) oil. In one embodiment, the moisturizing agent may be allantoin.

In certain embodiments, the cosmetic or dermatological formulations comprising the compositions as disclosed herein further comprise a preservative. In certain embodiments, the preservative is selected from one or more of quaternary ammonium preservatives such as polyquaternium-1 and benzalkonium halides (e.g., benzalkonium chloride (“BAC”) and benzalkonium bromide), parabens (e.g., methylparabens and propylparabens), phenoxyethanol, ethylhexylglycerin, ethylhexylglycerinbenzyl alcohol, chlorobutanol, phenol, sorbic acid, thimerosal and combinations thereof.

In certain embodiments, the cosmetic or dermatological formulations comprising the compositions as disclosed herein may further comprise excipients commonly used in the formulation of cosmetic or pharmaceutical preparations for topical use, such as bactericidal agents, stabilizers, emulsifiers, buffers, wetting agents, coloring agents, and other excipients commonly used in the cosmetic/pharmaceutical preparation techniques.

In certain embodiments, the cosmetic or dermatological formulations comprising the compositions as disclosed herein further comprise one or more emulsifiers. In certain such embodiments, the emulsifier reduces the interfacial tension between phases and improves the formulation and stability of an emulsion. The emulsifier may include a non-ionic emulsifier, an anionic emulsifier, a cationic emulsifier, a Zwitterionic emulsifier or a combination thereof. Non-limiting examples of emulsifiers include esters of glycerin, esters of propylene glycol, fatty acid esters of polyethylene glycol, fatty acid esters of polypropylene glycol, esters of sorbitol, esters of sorbitan anhydrides, carboxylic acid copolymers, esters and ethers of glucose, ethoxylated ethers, ethoxylated alcohols, alkyl phosphates, polyoxyethylene fatty ether phosphates, fatty acid amides, acyl lactylates, soaps, TEA stearate, DEA oleth-3 phosphate, polyethylene glycol 20 sorbitan monolaurate (polysorbate 20), polyethylene glycol 5 soya sterol, Steareth-2, Steareth-20, Steareth-21, ceteareth-20. PPG-2 methylglucose ether distearate, ceteth 10, polysorbate 80, cetyl phosphate, potassium cetyl phosphate, diethanolamine cetyl phosphate, polysorbate 60, glyceryl Stearate, PEG-100 stearate, and mixtures thereof. In one embodiment, the non-ionic emulsifier is cetearyl olivate or sorbitan olivate.

In certain embodiments, the compositions as disclosed herein are combined with one or more further cosmetic composition prior to use. In certain embodiments, the one or more further cosmetic composition comprises one or more agent selected from alpha and beta hydroxy acids, amino acids, peptides, matrix proteins, growth factors, stem cell activators, estrogens, anti-androgens, and skin lightening and brightening agents. In certain embodiments of the present application wherein the compositions as disclosed herein are combined with one or more further cosmetic composition prior to use, the one or more further cosmetic composition comprises one or more cosmetic ingredient. A wide variety of non-limiting cosmetic ingredients described in the CTFA International Cosmetic Ingredient Dictionary and Handbook (2004 and 2008) can be used. Non-limiting examples of cosmetic ingredients include fragrances (artificial and natural), dyes and color ingredients (e.g., Blue 1, Blue 1 Lake, Red 40, titanium dioxide, D&C blue no. 4, D&C green no. 5, D&C orange no. 4, D&C red no. 17, D&C red no. 33, D&C violet no. 2, D&C yellow no. 10, and D&C yellow no. 11), adsorbents, lubricants, solvents, moisturizers (including, e.g., emollients, humectants, film formers, occlusive agents, and agents that affect the natural moisturization mechanisms of the skin), water-repellants, UV absorbers (physical and chemical absorbers such as paraminobenzoic acid (“PABA”) and corresponding PABA derivatives, titanium dioxide, zinc oxide, etc.), essential oils, vitamins (e.g., A, B, C, D, E, and K), trace metals (e.g. zinc, calcium and selenium), anti-irritants (e.g. steroids and non-steroidal anti-inflammatories), botanical extracts (e.g. Aloe vera, chamomile, cucumber extract, Ginkgo biloba, ginseng, and rosemary), anti-microbial agents, antioxidants (e.g., BHT and tocopherol), chelating agents (e.g., disodium EDTA and tetrasodium EDTA), preservatives (e.g., methylparaben and propylparaben), pH adjusters (e.g., sodium hydroxide and citric acid), absorbents (e.g., aluminum starch octenylsuccinate, kaolin, corn starch, oat starch, cyclodextrin, talc, and zeolite), skin bleaching and lightening agents (e.g., hydroquinone and niacinamide lactate), humectants (e.g., sorbitol, urea, and manitol), exfoliants, waterproofing agents (e.g., magnesium/aluminum hydroxide stearate), skin conditioning agents (e.g., aloe extracts, allantoin, bisabolol, ceramides, dimethicone, hyaluronic acid, and dipotassium glycyrrhizate).

In certain embodiments of the present application, the compositions as disclosed herein are combined with one or more topical dermatologic drug composition prior to use. In certain such embodiments, the pharmaceutically active agent is selected from anti-acne agents, agents used to treat rosacea, analgesics, anesthetics, anorectals, antihistamines, anti-inflammatory agents including non-steroidal anti-inflammatory drugs, antibiotics, antifungals, antivirals, antimicrobials, anti-cancer actives, scabicides, pediculicides, antineoplastics, antiperspirants, antipruritics, antipsoriatic agents, antiseborrheic agents, biologically active proteins and peptides, burn treatment agents, cauterizing agents, depigmenting agents, depilatories, diaper rash treatment agents, enzymes, hair growth stimulants, hair growth retardants including eflornithine and its salts and analogs, hemostatics, kerotolytics, canker sore treatment agents, cold sore treatment agents, dental and periodontal treatment agents, photosensitizing actives, skin protectant/barrier agents, steroids including hormones and corticosteroids, sunburn treatment agents, sunscreens, transdermal actives, nasal actives, vaginal actives, wart treatment agents, wound treatment agents, wound healing agents, etc.

The foregoing is merely illustrative of the principles of the disclosure, and the apparatuses can be practiced by other than the described implementations, which are presented for purposes of illustration and not of limitation. Variations and modifications will occur to those of skill in the art after reviewing this disclosure. The disclosed features may be implemented, in any combination and subcombination (including multiple dependent combinations and subcombinations), with one or more other features described herein. The various features described or illustrated above, including any components thereof, may be combined or integrated in other systems, composition, and formulations. Moreover, certain features may be omitted or not implemented.

Examples of changes, substitutions, and alterations are ascertainable by one skilled in the art and could be made without departing from the scope of the information disclosed herein. All references cited herein are incorporated by reference in their entirety and made part of this application.

### EXEMPLIFICATION

The invention now being generally described, it will be more readily understood by reference to the following examples referring to the results documented in the accompanying Figures, which are included merely for purposes of illustration of certain aspects and embodiments of the present invention, and are not intended to limit the invention.

### Example 1: Antioxidant Properties of Xanthommatin

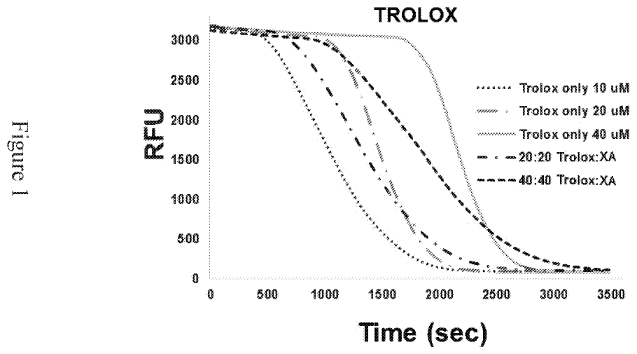

The antioxidant properties of ammonium xanthommatin (referred to herein as “xanthommatin”) in an unsaturated solution were compared to known standard antioxidants. The results of the experiments are provided in FIGS. 1-3, illustrating the relative fluorescence value (RFU) over time (in seconds) for known antioxidants Trolox (a vitamin E analogue) and ascorbic acid compared to xanthommatin. Experiments were performed using 10, 20, and 40 μM Trolox, ascorbic acid, and ammonium xanthommatin (“Xa”) and compared to performance of 1:1 Trolox:Xa (20 μM: 20 μM and 40 μM: 40 μM) and 1:1 ascorbic acid:Xa (20 μM: 20 μM and 40 μM: 40 μM).

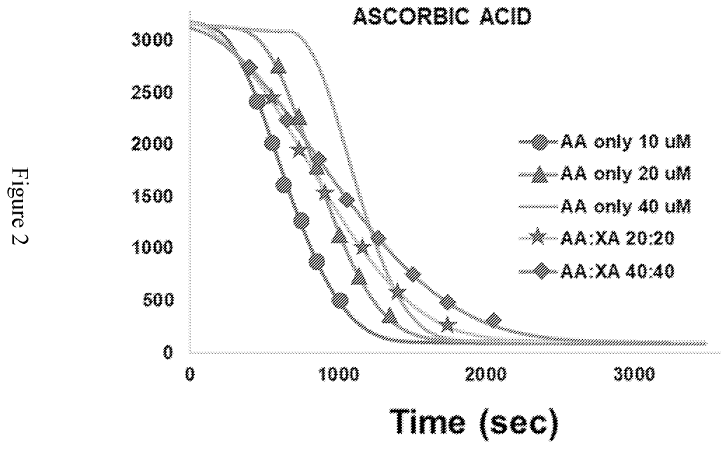

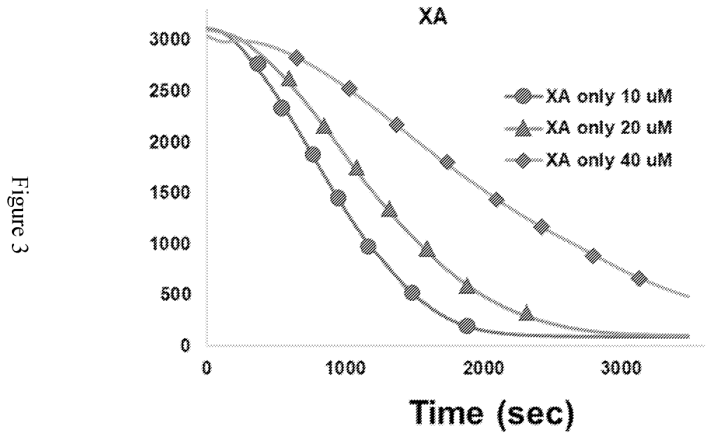

FIG. 1 illustrates the performance of Trolox and 1:1 Trolox:xanthommatin. FIG. 2 illustrates the performance of ascorbic acid and 1:1 ascorbic acid:xanthommatin. FIG. 3 illustrates xanthommatin alone. In all cases, the xanthommatin was in the form of unaggregated xanthommatin molecules in an unsaturated solution. FIG. 1 shows that a 1:1 ratio of Trolox to xanthommatin has a slower decrease in RFU over time as compared to compositions containing Trolox alone at concentrations of 20 μM or greater. The presence of xanthommatin in the composition delays degradation of the Trolox. Similarly, as illustrated in FIG. 2, the 1:1 ratio of ascorbic acid to xanthommatin in FIG. 3 maintains a higher RFU value for a longer period of time, resulting in a slower decrease in RFU value and delayed degradation compared to ascorbic acid alone at comparable concentrations. FIG. 3 shows that xanthommatin at concentrations of 10, 20 and 40 μM maintain a high RFU value for greater lengths of time compared to the compositions in FIGS. 1 and 2. Comparable activity level was observed in the compositions including xanthommatin as in compositions of antioxidants alone. Further, xanthommatin demonstrated the capability of delaying degradation of antioxidants over time to provide a longer lifetime when compared to compositions of known standard antioxidants alone.

Table 1 shows the results of calculation of the area under the curve (AUC) for the data presented in FIGS. 1-3.

TABLE 1
Comparisons of AUC

| | AUC |
|---|---|
| Trolox only, 40 uM | 55 |
| AA only, 40 uM | 55 |
| XA only, 40 uM | 56 |
| Trolox:XA 40:40 | 55 |
| Trolox:XA 20:20 | 50 |
| Ascorbic Acid:XA 40:40 | 54 |
| Ascorbic Acid:XA 20:20 | 55 |
| blank | 11 |

The results show that xanthommatin has comparable activity to vitamin c and vitamin e.

### Example 2: UV-Filter Stabilizing Properties of Xanthommatin

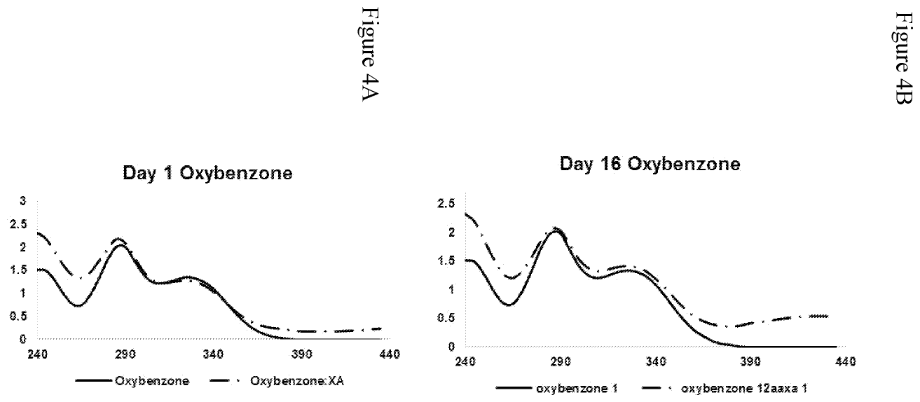

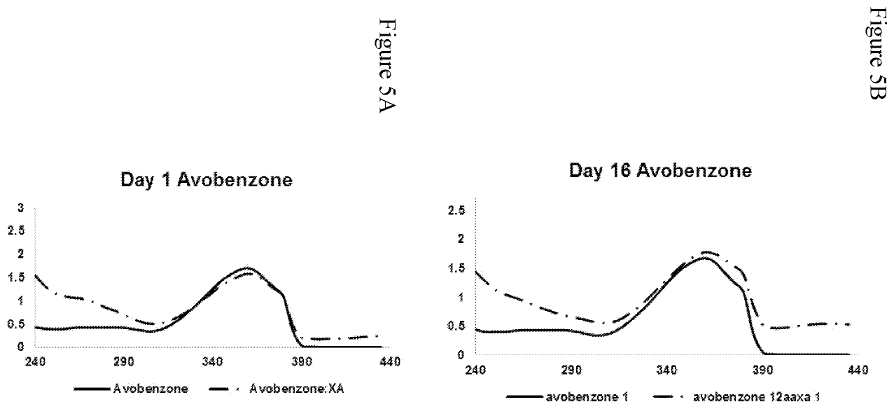

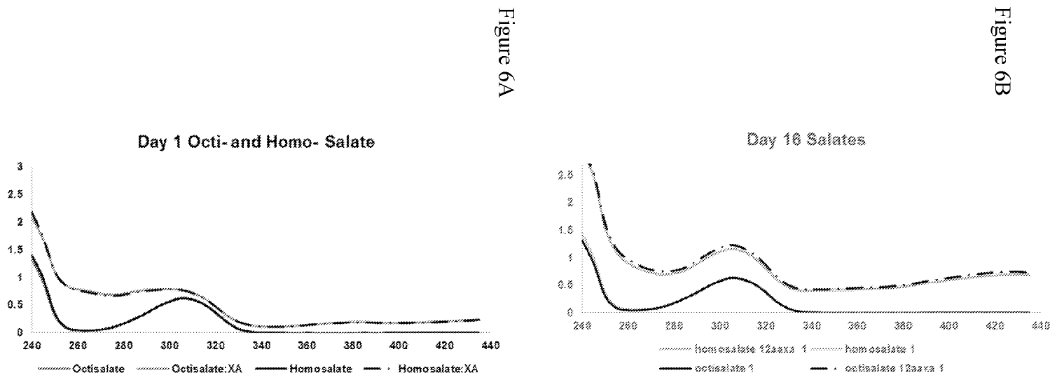

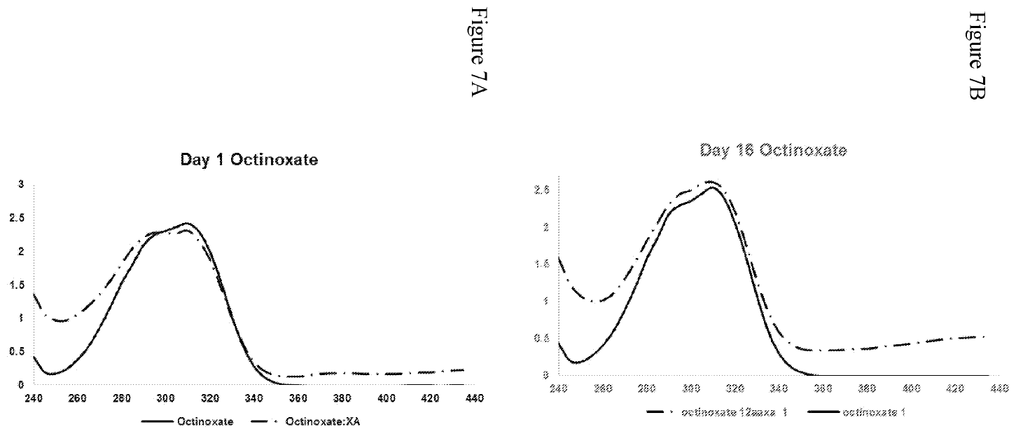

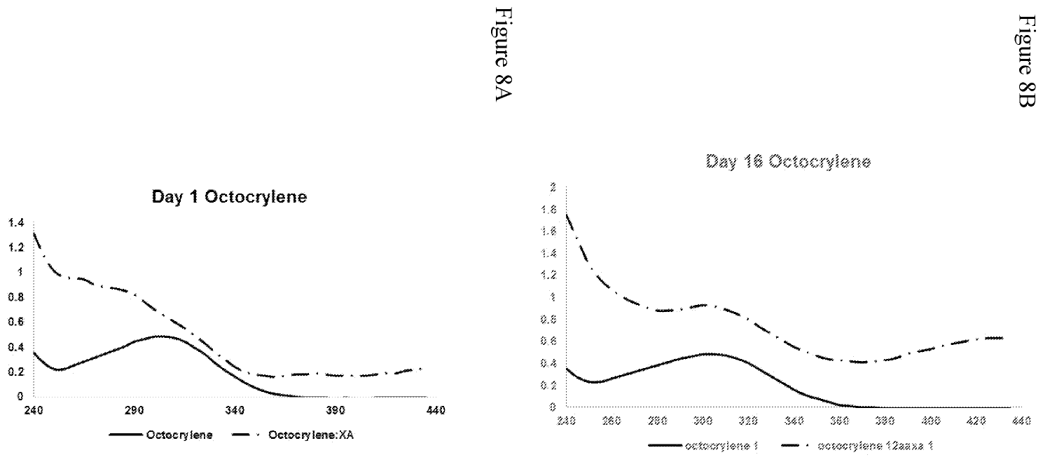

The UV-filter stabilizing properties of unsaturated solutions of xanthommatin were explored by comparing the behavior of UV-filter stabilizers over time to UV-filter stabilizers in combination with xanthommatin. As shown in FIGS. 4-8, the addition of unsaturated solutions of xanthommatin to solutions including oxybenzone (FIGS. 4A and 4B), avobenzone (FIGS. 5A and 5B), octi and homo-salate (FIGS. 6A and 6B), octinoxate (FIGS. 7A and 7B) and octocrylene (FIGS. 8A and 8B) increased the UV absorption between about 240 and about 310 nm on day 1, relative to the chemical UV filters alone. On day 16, the UV absorption profiles of the formulations including xanthommatin with the chemical UV filters are more similar to the day 1 UV absorption profiles and show higher UV absorption throughout the UV spectrum than the chemical UV filters alone. Accordingly, the presence of xanthommatin at about 0.01 wt % in a formulation with the chemical UV filters oxybenzone, avobenzone, octi- and homo-salates, octinoxate, and octocrylene enhances the UV absorption properties of the chemical UV filters and stabilizes the chemical UV filters over time.

### Example 3: UV-Boosting Properties of Xanthommatin

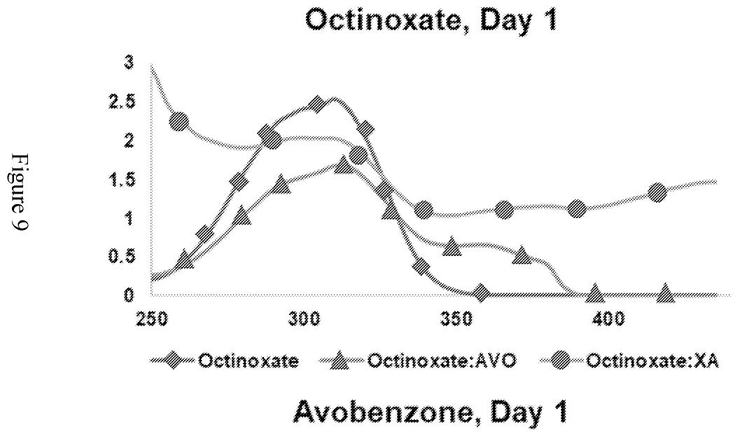

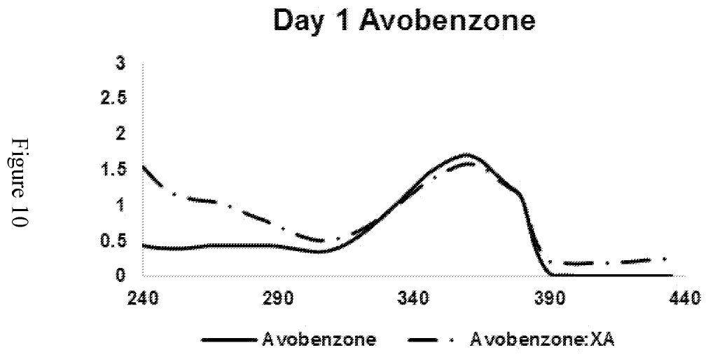

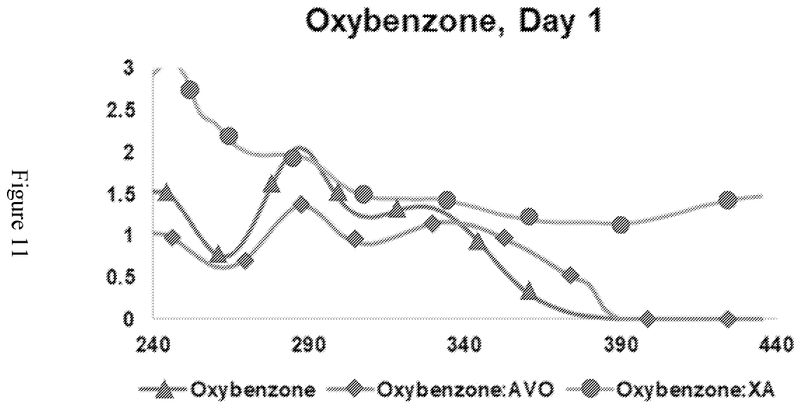

FIGS. 9-11 show the UV absorption behavior of chemical UV filters octinoxate, avobenzone, and oxybenzone, alone, in combination with avobenzone, and in combination with xanthommatin small molecules in unsaturated solution (0.03 wt %). Tables 2-4 show the changes in performance of the various combinations.

TABLE 2
Changes in UV absorption behavior
of octinoxate with xanthommatin

| | | Change in | | Change in |
|---|---|---|---|---|
| | | Performance | | Performance |
| | 310 nm | (%) | 360 nm | (%) |
| octinox control | 2.53 | | 0 | |
| octinox + Xa | 1.94 | −23 | 1.09 | 109% |

TABLE 3
Changes in UV absorption behavior of
avobenzone in various combinations

| | | Change in | | Change in |
|---|---|---|---|---|
| | | Performance | | Performance |
| | 300 nm | (%) | 360 nm | (%) |
| avo control | 0.31 | | 1.49 | |
| avo + Xa | 1.13 | 264 | 1.63 | 9 |
| avo + oxy | 1.02 | 229 | 0.79 | −46 |
| avo + octinox | 1.53 | 393 | 0.65 | −56 |

TABLE 4
Changes in UV absorption behavior
of oxybenzone with xanthommatin

| | | Change in | | Change in |
|---|---|---|---|---|
| | | Performance | | Performance |
| | 290 nm | (%) | 360 nm | (%) |
| Oxy control | 2 | | 0.31 | |
| Oxy + Xa | 1.78 | −11 | 1.19 | 283 |

The addition of xanthommatin to compositions including octinoxate, avobenzone, and oxybenzone alters the UV absorption profile of the composition and increases the UV absorption of the formulation across the UV spectrum. The combinations of octinoxate or oxybenzone with xanthommatin molecules show greater UV absorption than the same UV filter with avobenzone. The presence of xanthommatin at 0.03 wt % was shown to enhance the UVB absorbing power of octinoxate and oxybenzone by more than 250% in some cases. The presence of xanthommatin in the same amount boosted the performance of avobenzone by up to 109%. Accordingly, unsaturated solutions of xanthommatin are capable of functioning as a replacement to avobenzone and can also enhance UVB absorbing power of other filters.

### Example 4: Chemical and Mineral Sunscreens

Chemical sunscreens were prepared with ingredients as outlined in table 5.

TABLE 5
Chemical Sunscreens

| | | Xanthommatin |
|---|---|---|
| Active Ingredients | Control | Sample |
| Ethylhexyl | 7.0% | 7.0% |
| Methoxycinnamate | | |
| (Octinoxate) | | |
| Octocrylene | 10% | 10% |
| Homosalate | 15% | 15% |
| Avobenzone | 3.0% | 3.0% |
| Ammonium | 0% | 0.1% |
| Xanthommatin | | |

Specifically, the control chemical sunscreen shown above was prepared with the complete ingredient list provided in table 6 below. Phase A was prepared by solubilizing the disodium EDTA in water in the main beaker. The beaker was heated to 75° C. The solution was stirred and the C10-C30 Alkyl Acrylate Crosspolymer was slowly added and allowed to mix until the majority of the material was dispersed into solution. After the C10-C30 Alkyl Acrylate Crosspolymer was integrated, the solution was mixed until all the Crosspolymer was evenly dispersed throughout the mixture. Phase B was prepared by measuring Phase B ingredients 4-9 into a separate beaker and heating to 60° C. The mixture was stirred to ensure all the ingredients and waxes were melted and mixed. Phase B ingredients 10-12 were added to separate containers and slowly added to the Phase B Beaker allowing each ingredient to evenly disperse. When all were incorporated, phase B was added to phase A and mixed well. Agitation was increased to completely emulsify the batch. The batch was then cooled while continuing mixing. Phase C was added to the batch and mixed well to ensure integration. At 50° C. sodium hydroxide was added to the batch. The batch was then removed from the mixture and homogenized for 4 minutes. Mixing was then continued until the batch reached room temperature.

TABLE 6
Control Chemical Sunscreen

| | Phase | Ingredient Name | Functionality | Weight % |
|---|---|---|---|---|
| 1 | A | Deionized Water | | 42.980 |
| 2 | A | Disodium EDTA | Chelating Agent | 0.020 |
| 3 | A | Acrylates/C10-30 | Texture Enhancer | 0.300 |
| | | Alkyl Acrylate | | |
| | | Crosspolymer | | |
| 4 | B | Cetearyl Alcohol | Emulsifier | 5.000 |
| 5 | B | C12-15 Alkyl | Emollient | 8.000 |
| | | Benzoate | | |
| 6 | B | Isopropyl | Emollient | 5.000 |
| | | Myristate | | |
| 7 | B | Ethylhexyl | UV Filter | 7.000 |
| | | Methoxycinnamate | | |
| | | (Octinoxate) | | |
| 8 | B | Octocrylene | UV Filter | 10.000 |
| 9 | B | Homosalate | UV Filter | 15.000 |
| 10 | B | Avobenzone | UV Filter | 3.000 |
| 11 | B | Potassium Cetyl | Emulsifier | 1.200 |
| | | Phosphate | | |
| | | (Amphisol K) | | |
| 12 | B | Glyceryl Stearate | Emulsifier/Thickener | 1.000 |
| 13 | C | Phenoxyethanol | Preservative | 1.000 |
| 14 | D | Sodium Hydroxide | pH Adjuster | 0.500 |
| | | 30% Soln | | |
| | | Total | | 100.000 |

The chemical sunscreen containing ammonium xanthommatin shown above was prepared with the complete ingredient list provided in table 7 below. Specifically, phase A was prepared by solubilizing the disodium EDTA in water in the main beaker and heating to 75 AC. The solution was stirred and C10-C30 Alkyl Acrylate Crosspolymer was slowly added and allowed to mix until the majority of the material was dispersed into solution. After the C10-C30 Alkyl Acrylate Crosspolymer was integrated, the ammonium xanthommatin was added and mixed until dissolved and the Crosspolymer was evenly dispersed throughout the mixture. Phase B ingredients 5-10 were added to a separate beaker, heated to 60° C., and stirred to ensure all the ingredients and waxes were melted and mixed. The mixture was allowed to continue stirring. Phase B ingredients 11-13 were slowly added separately to the Phase B Beaker, allowing each ingredient to evenly disperse. When all were incorporated, phase B was added to phase A and mixed well. Agitation was increased to completely emulsify the batch before cooling the batch while continuing mixing. Phase C was then added to the batch mix well to ensure integration. Sodium hydroxide was then added at 50° C. and the batch then removed from the mixer and homogenizes for 4 minutes before allowing to continue mixing until the batch reached room temperature.

TABLE 7
Chemical Sunscreen Containing Ammonium Xanthommatin

| | Phase | Ingredient Name | Functionality | Weight % |
|---|---|---|---|---|
| 1 | A | Deionized Water | | 42.880 |
| 2 | A | Disodium EDTA | Chelating Agent | 0.020 |
| 3 | A | Acrylates/C10-30 | Texture Enhancer | 0.300 |
| | | Alkyl Acrylate | | |
| | | Crosspolymer | | |
| 4 | A | Xanthommatin | UV Booster | 0.100 |
| 5 | B | Cetearyl Alcohol | Emulsifier | 5.000 |
| 6 | B | C12-15 Alkyl | Emollient | 8.000 |
| | | Benzoate | | |
| 7 | B | Isopropyl | Emollient | 5.000 |
| | | Myristate | | |
| 8 | B | Ethylhexyl | UV Filter | 7.000 |
| | | Methoxycinnamate | | |
| | | (Octinoxate) | | |
| 9 | B | Octocrylene | UV Filter | 10.000 |
| 10 | B | Homosalate | UV Filter | 15.000 |
| 11 | B | Avobenzone | UV Filter | 3.000 |
| 12 | B | Potassium Cetyl | Emulsifier | 1.200 |
| | | Phosphate | | |
| | | (Amphisol K) | | |
| 13 | B | Glyceryl Stearate | Emulsifier/Thickener | 1.000 |
| 14 | C | Phenoxyethanol | Preservative | 1.000 |
| 15 | D | Sodium Hydroxide | pH Adjuster | 0.500 |
| | | 30% Soln | | |
| | | Total | | 100.000 |

Mineral sunscreens were prepared with ingredients as outlined in table 8.

TABLE 8
Mineral Sunscreens

| | | Xanthommatin |
|---|---|---|
| Active Ingredients | Control | Sample |
| Titanium Dioxide | 7.0% | 8.0% |
| Zinc Oxide | 10% | 10% |
| Xanthommatin | 0% | 0.1% |

Specifically, the control mineral sunscreen shown above was prepared with the complete ingredient list provided in table 9 below. Phase A was prepared by solubilizing the disodium EDTA in water in the main beaker and heating the beaker to 75° C. with moderate mixing. In a separate beaker, glycerin, Xantham Gum, and hydroxyethylcellulose were combined and mixed well before adding to the main beaker. The batch was allowed to stir for approximately ˜5 min. Phase B ingredients 6-11 were added to a separate beaker, heated to 75° C., and stirred to ensure all the ingredients and waxes were melted and mixed. This beaker was allowed to stir with moderate mixing before adding Phase B ingredient 14. Phase B ingredient 12 was then added to the batch portionwise, allowing the material to disperse into the oil phase before adding the next portion. When all of ingredient 12 was added, the same process was repeated with ingredient 13. Once both ingredients were incorporated and mixed into phase B, phase B was added to phase A and mixed well before increasing the agitation to completely emulsify the batch. Once the batch emulsified under heat, the beaker was removed from the hot plate and homogenized continuously and thoroughly under heat for 5 minutes. The resulting mixture was then allowed to continue stirring with cooling to room temperature.

TABLE 9
Control Mineral Sunscreen

| | Phase | Ingredient Name | Functionality | Weight % |
|---|---|---|---|---|
| 1 | A | Deionized Water | | 50.370 |
| 2 | A | Disodium EDTA | Chelating Agent | 0.030 |
| 3 | A | Glycerin | Humectant | 3.000 |
| 4 | A | Hydroxethylcellulose | Thickener | 0.000 |
| 5 | A | Xanthan Gum | Thickener | 0.500 |
| 6 | B | Cetearyl Alcohol | Emulsifier | 3.000 |
| 7 | B | C12-15 Alkyl Benzoate | Emollient | 10.000 |
| 8 | B | Isododecane | Emollient | 5.000 |
| 9 | B | Isopropyl Myristate | Emollient | 5.000 |
| 10 | B | Cream Maker Fluid | Emulsifier/Thickener | 4.000 |
| 11 | B | Glyceryl Stearate Citrate | Emulsifier/Thickener | 0.100 |
| 12 | B | Titanium Dioxide | UV Filter | 8.000 |
| 13 | B | Zinc Oxide | UV Filter | 10.000 |
| 14 | B | Phenoxyethanol & | Preservative | 1.000 |
| | | hexylglycerin (EUXYL | | |
| | | 9010) | | |
| | | Total | | 100.000 |

The mineral sunscreen containing ammonium xanthommatin shown above was prepared with the complete ingredient list provided in table 10 below. Phase A was prepared by solubilizing the disodium EDTA in water in the main beaker and heating the beaker to 75° C. with moderate mixing. Ammonium xanthommatin was then added in batches, allowing mixing between each addition to allow the ammonium xanthommatin to dissolve into the mixture. Once complete, the mixture was cooled to 40° C. and NaOH added as needed to reach a pH of 5.0 to 5.5. The mixture was then heated to 75° C. with moderate mixing. In a separate beaker, glycerin and Xantham Gum, were combined and mixed well before adding to the main beaker. The batch was allowed to stir for approximately 10 min. Phase B ingredients 7-12 were added to a separate beaker, heated to 75° C., and stirred to ensure all the ingredients and waxes were melted and mixed. This beaker was allowed to stir with moderate mixing before adding Phase B ingredient 13 to the batch portionwise, allowing the material to disperse into the oil phase before adding the next portion. When all of ingredient 13 was added, the same process was repeated with ingredient 14. Once both ingredients were incorporated and mixed into phase B, ingredient 15 was added to phase B, and then phase B was added to phase A and mixed well before increasing the agitation to completely emulsify the batch. Once the batch emulsified under heat, the beaker was removed from the hot plate and homogenized continuously and thoroughly under heat for 5 minutes. The resulting mixture was then allowed to continue stirring with cooling to room temperature. QS with water.

TABLE 10
Mineral Sunscreen Containing Ammonium Xanthommatin

| | Phase | Ingredient Name | Functionality | Weight % |
|---|---|---|---|---|
| 1 | A | Deionized Water | | 49.142 |
| 2 | A | Disodium EDTA | Chelating Agent | 0.030 |
| 3 | A | Xanthommatin | UV Filter/Booster | 0.100 |
| 4 | A | 30% NaOH Solution | pH Adjustment | 0.888 |
| 5 | A | Glycerin | Humectant | 3.000 |
| 6 | A | Xanthan Gum | Thickener | 0.500 |
| 7 | B | Cetearyl Alcohol | Emulsifier | 3.000 |
| 8 | B | C12-15 Alkyl Benzoate | Emollient | 10.000 |
| 9 | B | Isododecane | Emollient | 5.000 |
| 10 | B | Isopropyl Myristate | Emollient | 5.000 |
| 11 | B | Cream Maker Fluid | Emulsifier/Thickener | 4.000 |
| 12 | B | Glyceryl Stearate Citrate | Emulsifier/Thickener | 0.100 |
| 13 | B | Titanium Dioxide | UV Filter | 8.000 |
| 14 | B | Zinc Oxide | UV Filter | 10.000 |
| 15 | B | Euxyl PE 9010 | Preservative | 1.000 |
| 16 | C | 50% Citric Acid Solution | pH Adjustment | 0.240 |
| | | Total | | 100.000 |

The Labsphere UV 2000S Ultraviolet Transmittance Analyzer or Solar Light Company, Inc. SPF290-AS was used to determine spectral transmittance for each wavelength over the full UV spectrum (290 to 400 nanometers). The transmittance values were measured at 1 nanometer intervals. Solar Light Model LS 1000-4S-009 was used for UV irradiation. The simulator was fitted with UV dichroic mirror, WG320 filter, heat filter, and UG5 filter to provide a continuous emission spectrum from 290-400 nm with a limit of 1,500 Watts/m2 on total solar simulator irradiance for all wavelengths between 250 and 400 nm. The percentage of erythema effective radiation in each specified wavelength is shown in Table 11

TABLE 11
Percentage of Erythema Effective
Radiation at Various Wavelengths

| Wavelength range (nm) | Erythemal Contribution (%) |
|---|---|
| <290 | <0.1 |
| 290-300 | 1.0-8.0 |
| 290-310 | 49.0-65.0 |
| 290-320 | 85.0-90.0 |
| 290-330 | 91.5-95.5 |
| 290-340 | 94.0-97.0 |
| 290-400 | 99.9-100.0 |

Sunscreen was applied to four new, untreated roughened PMMA plate (with the roughened side uppermost) at an application rate of 1.3 mg/cm2. The sunscreen was applied as a large number of small droplets approximately equal in volume, distributed equally over the whole surface of the plate. Using a fingercot, the product was distributed using a very light spreading action for approximately 30 seconds followed by spreading with greater pressure for approximately 30 seconds. The sample was allowed to dry for 30 minutes protected from light in a controlled temperature (25-35° C.). 2. Five spectra of the product on different points of the PMMA plate were obtained, taken at 1 nm intervals in the 290-400 nm range. The PMMA plates containing the sample were exposed to a controlled dose of UV radiation to simulate four hours of sun (UV) exposure. Following irradiation, five spectra of the product on different points of the PMMA plate were again obtained, taken at 1 nm intervals in the 290-400 nm range. Reference (blank) measurements of a PMMA treated with glycerin were performed. Initial and final absorbance in the total UV (290-400 nm), UVB (290-320 nm), and UVA (320-400 nm) ranges were calculated. Percentage of stability was calculated by the ratio between the final value (after irradiation) and the initial value (before irradiation).

SPF was calculated using the derived Mansur equation (Sayre, R. M., et al., A comparison of in vivo and in vitro testing of sunscreening formulas. Photochemistry and Photobiology, 1979. 29(3): p. 559-566) according to Equation 1, SPF=CFΣ₂₉₀³²⁰EE(λ)I(λ)Abs(λ), where EE (λ) represents the erythemal effect spectrum; I(λ) is solar intensity spectrum; Abs(a) absorbance of UV-filter; CF is the correction factor (=10). The values for the normalized product function [EE(λ)×I(λ)] used in the calculation of SPF can be found in Table 12. The calculated SPF values were rounded to the nearest whole number. The critical wavelength was determined by applying this data to Equation 2, ∫₂₉₀^{λc}A(λ)dλ=0.9∫₂₉₀⁴⁰⁰A(λ)dλ, where A(λ) represents the absorbance of UV-absorbing compound and λ_c is the critical wavelength. (Administration, U.S.F.D., Code of Federal Regulations Title 21: Sunscreen drug products for over-the-counter human use U.S.D.o.H.H. Services, Editor. 2018.)

TABLE 12
Normalized product function used in the calculation of SPF

| Wavelength (nm) | EE × I (normalized) |
|---|---|
| 290 | 0.0150 |
| 295 | 0.0817 |
| 300 | 0.2874 |
| 305 | 0.3278 |
| 310 | 0.1864 |
| 315 | 0.0839 |
| 320 | 0.0180 |
| Total | 1 |

The photostability was determined by applying the above data to Equations 3, AUC=∫₂₉₀⁴⁰⁰A(λ)dλ, and 4,

$$AUC Index ( AUCI ) = \frac{{AUC}_{t}}{{AUC}_{i = n}} ,$$

where AUC is the area under the curve derived from the UV-absorbance spectra from 290-400 nm calculated using Equation 3; AUCt=0 is the area under the curve determined prior to irradiation; AUCt is the area under the curve at a specific time point (e.g. 30, 60, and 90 mins); AUC Index (AUCI) is the quotient of AUC after irradiation and AUC before irradiation calculated from Equation 4. A compound or material is considered to be photo-stable if AUCI>0.8. (Hojerova, J., Medovcikova, A. and M. Mikula, Photoprotective efficacy and photostability of fifteen sunscreen products having the same label SPF subjected to natural sunlight. International Journal of Pharmaceutics, 2011. 408: p. 27-38.)

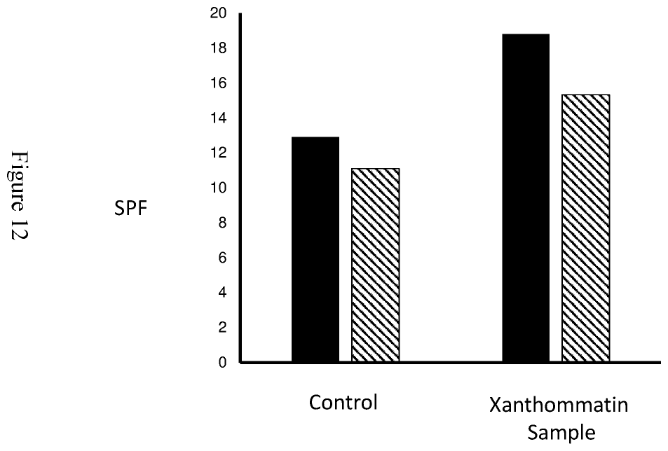

The results from measuring the SPF of the chemical sunscreen samples pre- and post-irradiation are provided in FIG. 12. In this figure, solid bars are pre-irradiation measurements, and patterned bars are post-irradiation measurements. As shown, the chemical sunscreen containing 0.1% ammonium xanthommatin provided a boost in SPF by 45% pre-irradiation and 38% post-irradiation.

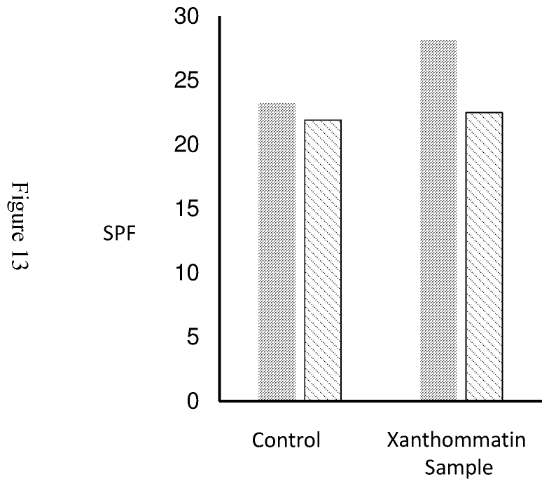

The results from measuring the SPF of the mineral sunscreen samples pre- and post-irradiation are provided in FIG. 13. In this figure, solid bars are pre-irradiation measurements, and patterned bars are post-irradiation measurements. As shown, the mineral sunscreen containing 0.1% ammonium xanthommatin provided a boost in SPF by 21% pre-irradiation and 3% post-irradiation.

The performance of both the chemical and mineral sunscreens including ammonium xanthommatin can also be seen from the data provided in tables 11 below. Specifically, as can be seen in table 13, the addition of 0.1% ammonium xanthommatin to both mineral and chemical sunscreens boosted the SPF and UVA PF of the formulation.

TABLE 13
Performance of Ammonium Xanthommatin
in Chemical and Mineral Sunscreens

| | | | Critical | Critical | | |
|---|---|---|---|---|---|---|
| | Mean | Mean | λ₀ | λᵢᵣᵣ | UVA | UVA |
| Formulation | SPF₀ | SPFᵢᵣᵣ | (nm) | (nm) | PF₀ | PFᵢᵣᵣ |
| chemical control | 12.91 | 11.10 | 377.18 | 371.09 | 6.77 | 4.93 |
| chemical + Xa | 18.80 | 15.33 | 379.39 | 374.30 | 17.44 | 13.76 |
| mineral control | 23.24 | 21.92 | 378.64 | 378.56 | 10.78 | 10.33 |
| mineral + Xa | 28.18 | 22.50 | 379.06 | 379.11 | 13.48 | 11.59 |

The post-irradiation versus pre-irradiation stability of both chemical and mineral sunscreens including ammonium xanthommatin can be seen from the data provided in table 14 below. Specifically, SPF stability of the control chemical sunscreen formulations was improved with 0.1% ammonium xanthommatin.

TABLE 14
Stability of Ammonium Xanthommatin
Chemical and Mineral Sunscreens

| | UVA | UVB | |
|---|---|---|---|
| | Absorbance | Absorbance | Total UV |
| Formulation | Stability (%) | Stability (%) | Stability (%) |
| chemical control | 77.65 | 97.86 | 84.90 |
| chemical + Xa | 80.90 | 95.07% | 85.59 |
| mineral control | 98.32 | 98.04 | 98.21 |
| mineral + Xa | 94.66 | 92.59 | 93.92 |

### Example 5: Absorbance and Cytotoxicity Studies

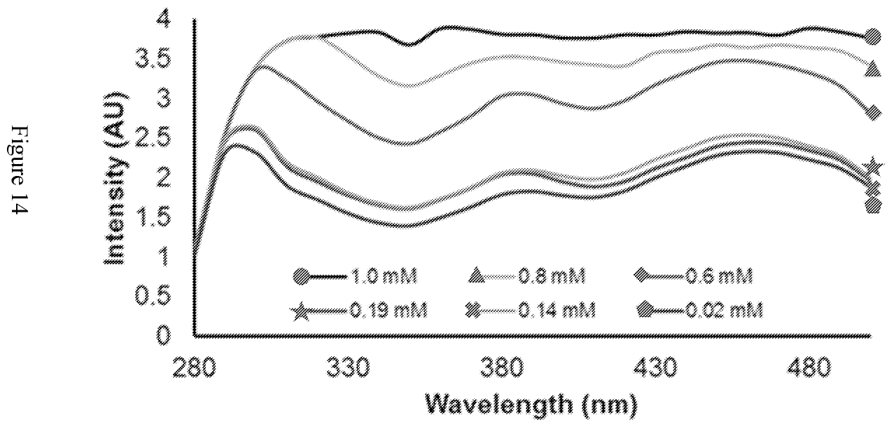

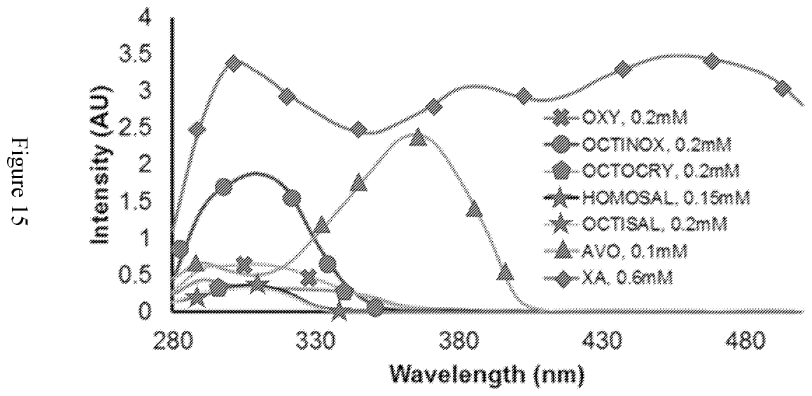

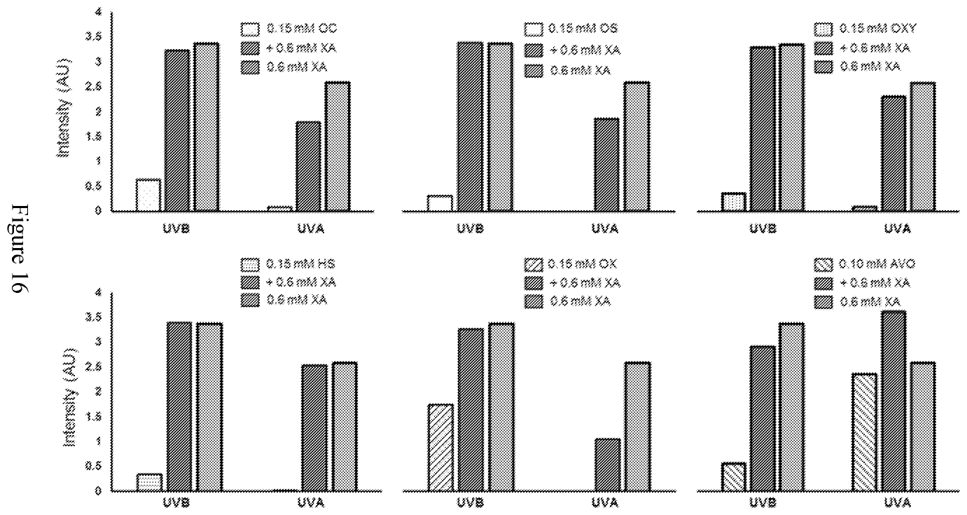

The performance of ammonium xanthommatin was tested as a broad-spectrum absorber to assess whether the natural optical features of xanthommatin could be used to boost the UV performance of low (<0.2 mM) concentrations of organic UV filters. Xanthommatin's absorbance capabilities were tested alone in solution (FIG. 14) and in combination with FDA approved organic UV filters (FIGS. 15 and 16) over a spectral range of 280-500 nm. We observed a clear relationship between increasing concentrations of xanthommatin and the absorption of UV through visible light (FIG. 14). Specifically, when compared to the absorptive behaviors of the pure organic UV filters (0.1-0.2 mM in DMSO), xanthommatin (0.6 mM in DMSO) exhibited a broader profile that spanned the UVB through visible light regions. When xanthommatin was combined with the chemical UV filters, a significant increase in both the UVB (300 nm) and UVA (360 nm) range was achieved across all filters tested, where we observed at least a 50% increase in UVA and B performance upon addition with xanthommatin (FIG. 16). These results effectively demonstrate that xanthommatin boosts the absorption profiles of these molecules in solution.

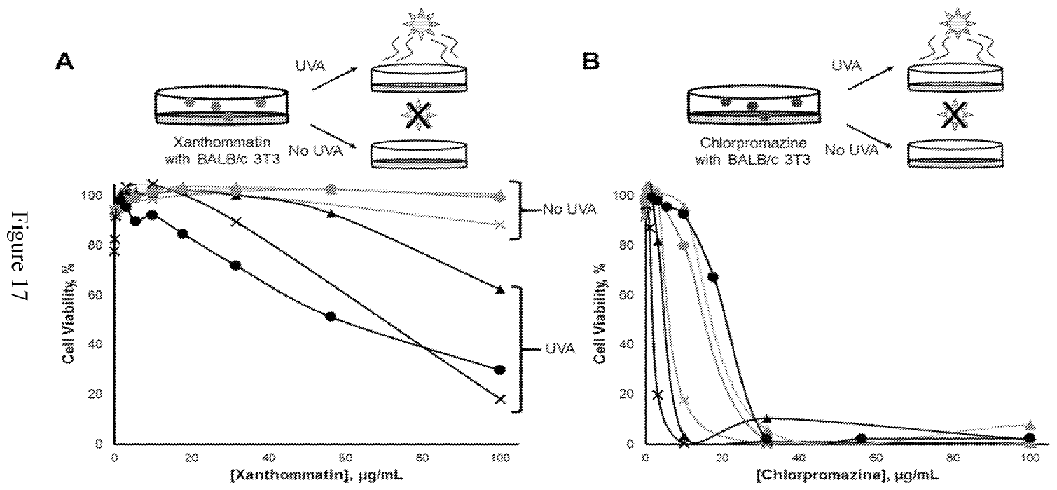

Given the UV filter-boosting features of xanthommatin, we next tested its cytotoxicity with and without exposure to a noncytotoxic dose of UVA light (from 315 to 400 nm and a measuring range between 0-199.9 mW/cm2). In these experiments, cytotoxicity was expressed as a concentration dependent reduction of a neutral red dye uptake within BALB/c 3T3 mouse fibroblast cell lines after treatment both with and without the presence of UVA exposure. (Borenfreund, E. and J. A. Puerner, Toxicity determined in vitro by morphological alterations and neutral red absorption. Toxicol Lett, 1985. 24(2-3): p. 119-24.) In the presence of UVA, the cells treated with xanthommatin showed clear cytotoxic effects, where the relative cell viability at the highest test item concentration across three independent +UVA experiments was 18.1%, 29.7%, and 62.3% compared to the −UVA controls (88.5%, 99.3%, and 100.6% for the 3 experiments, FIG. 17A). Because no EC50-value could be calculated for the −UVA experiments, a photo-irritation-factor (PIF=EC50 (−UVA)/EC50(+UVA)) could not be calculated. Instead, the mean photo effect was calculated using Equation 5,

$$( MPE = \frac{{\sum }_{i = 1}^{n} {w}_{i} {PE}_{Ci}}{{\sum }_{i = 1}^{n} {w}_{i}} ) ;$$

where, the photo effect (PE) at any concentration (C) is defined as the product of the response effect (REC) and the dose effect (DEC) concentrations. MPE values of 0.019, 0.199, and 0.042 were measured for the three independent experiments with xanthommatin. An MPE>0.15 indicates phototoxicity; thus, xanthommatin was classified as not phototoxic because two of the three datasets below this threshold. In this figure, the grey lines represent data collected in the absence of UVA (−UVA), and the black lines represent data collected with exposure to UVA (+UVA). Data was collected for three independent experiments.

The controls confirmed the validity of the study, wherein negative controls of the +UVA experiments exhibited cell viabilities of 86.80% (exp. 1), 92.03% (exp. 2) and 102.39% (exp. 3) relative to the untreated, −UVA controls. On the other hand, the calculated EC50 values of the positive controls containing chlorpromazine for the −UVA (8.327 μg/mL exp. 1, 15.530 μg/mL exp. 2, and 18.809 μg/mL exp. 3) and the +UVA experiment (0.221 g/mL exp. 1, 0.413 μg/mL exp. 2, and 0469 μg/mL exp. 3) were within the validity ranges. The PIF values for the positive controls were 37.81 exp. 1, 37.74 exp. 2, and 40.33 exp. 3 (FIG. 17B).

Since the toxicological endpoint of this in vitro 3T3 NRU phototoxicity assay was developed and validated in a joint EU/COLIPA project, this assay is a well-recognized in vitro alternative to the various in vivo tests in use. (Spielmann, H., et al., EEC/COLIPA project on in vitro phototoxicity testing: First results obtained with a Balb/c 3T3 cell phototoxicity assay. Toxicol In Vitro, 1994. 8(4): p. 793-6; Balls, M. Statement on the scientific validity of the 3T3 NRU PT test (an in vitro test for phototoxic potential). 1998; Spielmann, H., et al., The International EU/COLIPA In Vitro Phototoxicity Validation Study: Results of Phase II (Blind Trial). Part 1: The 3T3 NRU Phototoxicity Test. Toxicol In Vitro, 1998. 12(3): p. 305-27.) According to these guidelines, our data infers that xanthommatin is neither phototoxic nor a photoirritant, where phototoxicity is defined as a toxic response that occurs either after the first exposure to the test chemicals followed by subsequent exposure to light or induced after systemic administration of a chemical after irradiation.

### Example 6: Antioxidant Stabilizing/Boosting

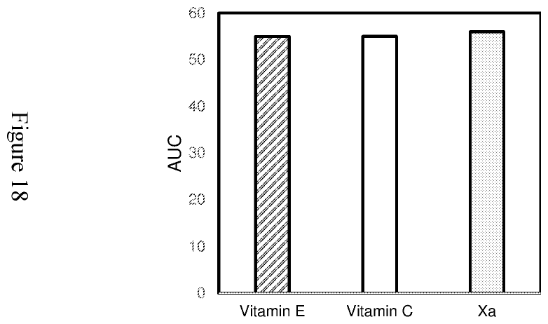

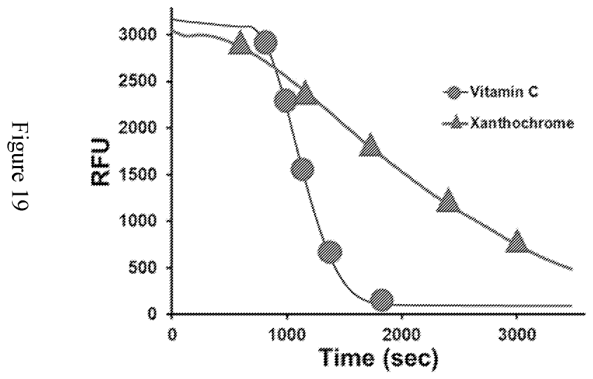

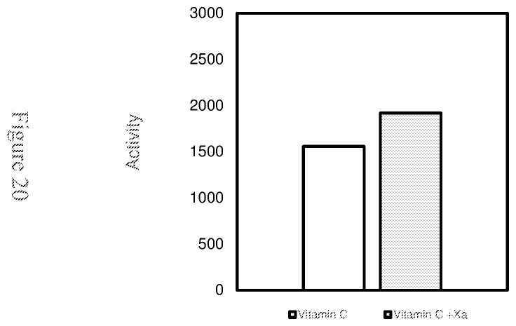

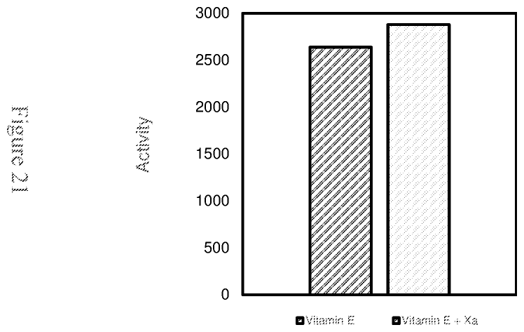

For antioxidant stabilizing/boosting experiments, the antioxidizing capacity and performance of ammonium xanthommatin only and ammonium xanthommatin blended with equimolar ascorbic acid (vitamin C) or Trolox (a vitamin E analogue) over 90 minutes at 37° C. were tested using a standard Oxygen Radical Antioxidant Capacity (ORAC) Assay. The ORAC assay measured the antioxidant capacity of biomolecules based on the oxidation of a fluorescein probe by peroxyl radicals (Fluorescence decreases as antioxidant capacity decreases). The ORAC assay is described in detail at https://www.cellbiolabs.com/sites/default/files/STA-345-orac-assay-kit.pdf, the contents of which are incorporated by reference herein. In all cases, ammonium xanthommatin provided comparable antioxidizing capacity to known antioxidant standards (Trolox and vitamin C). These findings were supported by the comparable area under the curve (AUC) calculations for the highest concentration (40 uM) conditions (FIG. 18). FIG. 19 provides half life measurements of ammonium xanthommatin in solution compared to vitamin C in solution when assayed at the same concentration. The solution of ammonium xanthommatin in solution was two times longer than vitamin C when assayed at the same concentration. FIGS. 20 and 21, respectively, show the performance of vitamin C and vitamin E is extended when combined with ammonium xanthommatin. Specifically, FIG. 20 shows that the performance of vitamin C was extended by 23% when combined with ammonium xanthommatin. FIG. 21 shows that the performance of vitamin E was extended by 9% when combined with ammonium xanthommatin.

1. A composition comprising:
at least one UV-filtering material; and
an unsaturated solution of 1 wt % or less of one or more phenoxazone and/or phenoxazine compound, a precursor or derivative thereof, or a salt of any of the foregoing.

2. The composition of claim 1, wherein the at least one UV-filtering material and the unsaturated solution of 1 wt % or less of one or more phenoxazone and/or phenoxazine compound, a precursor or derivative thereof, or a salt of any of the foregoing together exhibit synergy.

3. The composition of claim 1 or 2, wherein the composition has a pre-irradiation SPF greater than a composition comprising the at least one UV-filtering material without the unsaturated solution of 1 wt % or less of one or more phenoxazone and/or phenoxazine compound, a precursor or derivative thereof, or a salt of any of the foregoing.

4. The composition of any of claims 1-3, wherein the composition has a post-irradiation SPF greater than a composition comprising the at least one UV-filtering material without the unsaturated solution of 1 wt % or less of one or more phenoxazone and/or phenoxazine compound, a precursor or derivative thereof, or a salt of any of the foregoing.

5. The composition of claim 3 or 4, wherein the composition has an increase in SPF of greater than 10% as compared to a composition comprising the at least one UV-filtering material without the unsaturated solution of 1 wt % or less of one or more phenoxazone and/or phenoxazine compound, a precursor or derivative thereof, or a salt of any of the foregoing.

6. The composition of claim 3 or 4, wherein the composition has an increase in SPF of greater than 25% as compared to a composition comprising the at least one UV-filtering material without the unsaturated solution of 1 wt % or less of one or more phenoxazone and/or phenoxazine compound, a precursor or derivative thereof, or a salt of any of the foregoing.

7. The composition of any preceding claim, wherein the SPF of the composition is maintained for at least one week.

8. The composition of any preceding claim, wherein the SPF of the composition is maintained for at least two weeks.

9. The composition of any preceding claim, wherein the SPF of the composition is maintained for at least three weeks.

10. The composition of any preceding claim, wherein the composition exhibits greater UV absorbance than a composition comprising the at least one UV-filtering material without the unsaturated solution of 1 wt % or less of one or more phenoxazone and/or phenoxazine compound, a precursor or derivative thereof, or a salt of any of the foregoing.

11. The composition of any preceding claim, wherein the composition exhibits a change in UV absorbance of greater than 100% as compared to a composition comprising the at least one UV-filtering material without the unsaturated solution of 1 wt % or less of one or more phenoxazone and/or phenoxazine compound, a precursor or derivative thereof, or a salt of any of the foregoing.

12. The composition of any preceding claim, wherein the composition exhibits a change in UV absorbance of greater than 150% as compared to a composition comprising the at least one UV-filtering material without the unsaturated solution of 1 wt % or less of one or more phenoxazone and/or phenoxazine compound, a precursor or derivative thereof, or a salt of any of the foregoing.

13. The composition of any preceding claim, wherein the composition exhibits a change in UV absorbance of greater than 200% as compared to a composition comprising the at least one UV-filtering material without the unsaturated solution of 1 wt % or less of one or more phenoxazone and/or phenoxazine compound, a precursor or derivative thereof, or a salt of any of the foregoing.

14. The composition of any preceding claim, wherein the one or more phenoxazone and/or phenoxazine compound, a precursor or derivative thereof, or a salt of any of the foregoing comprises xanthommatin, decarboxylated xanthommatin, uncyclized xanthommatin, ommatin D, dihydroxy-xanthommatin, or rhodommatin, a derivative or precursor thereof, or a salt of any of the foregoing.

15. The composition of claim 14, wherein the one or more phenoxazone and/or phenoxazine compound, a precursor or derivative thereof, or a salt of any of the foregoing comprises xanthommatin, or a salt thereof.

16. The composition of any preceding claim, wherein the unsaturated solution of one or more phenoxazone and/or phenoxazine compound, a precursor or derivative thereof, or a salt of any of the foregoing comprises unaggregated molecules.

17. The composition of any preceding claim, wherein the at least one UV-filtering material comprises at least one UV-absorbing compound.

18. The composition of claim 17, wherein the at least one UV-absorbing compound is selected from avobenzone, oxybenzone, oxybenzone cinoxate, homosalate, octisalate, octinoxate, octocrylene, and trolamine salicylate.

19. The composition of any of claims 1-16, wherein the at least one UV-filtering material comprises at least one UV-scattering particle.

20. The composition of claim 19, wherein the at least one UV-scattering particle is selected from titanium dioxide and zinc dioxide.

21. The composition of any preceding claim, wherein the composition is formulated as a solution.

22. The composition of claim 21, wherein the at least one UV-filtering material and the unsaturated solution of one or more phenoxazone and/or phenoxazine compound, a precursor or derivative thereof, or a salt of any of the foregoing are homogeneously distributed within the solution.

23. The composition of any of claims 1-18, wherein the composition is formulated as an emulsion.

24. The composition of any preceding claim, further comprising one or more non-ionic polymeric emulsifier selected from potassium cetyl phosphate, PEG-150 distearate, cetearyl alcohol, caprylic/capric triglyceride, and glyceryl stearate.

25. The composition of any preceding claim, wherein the composition is formulated as a cream, gel, spray, or lotion for use in a cosmetic or dermatological formulation.

26. The composition of any preceding claim, wherein the composition is formulated to provide protection from solar ultraviolet radiation.

27. The composition of any preceding claim, wherein the composition is formulated to provide SPF of at least 15.

28. The composition of any of claims 1-26, wherein the composition is formulated to provide SPF of at least 30.

29. The composition of any of claims 1-26, wherein the composition is formulated to provide SPF of 15-100

30. The composition of any of claims 1-26, wherein the composition is formulated to provide SPF of 60 or more.

31. The composition of any of claims 1-30, wherein the composition is formulated to provide UVA-PF of at least 15.

32. The composition of any of claims 1-30, wherein the composition is formulated to provide UVA-PF of at least 30.

33. The composition of any of claims 1-30, wherein the composition is formulated to provide UVA-PF of 15-100.

34. The composition of any of claims 1-30, wherein the composition is formulated to provide UVA-PF of 60 or more.

35. The composition of any of claims 1-34, wherein the composition is formulated to provide broad spectrum protection.

36. The composition of any of claims 1-35, wherein the composition is formulated to provide a ratio of UVA I to UV B filters of at least 0.7 or greater.

37. The composition of any preceding claim, further comprising at least one additional anti-oxidizing compound.

38. The composition of claim 37, wherein the at least one additional anti-oxidizing compound comprises one or more of arbutin, BHA, BHT, koji acid, hydroxyanisole, hydroquinone, t-butyl hydroquinone, tocopherol, nordihydroguaiaretic acid, rosmarinic acid, Trolox, goosypol, flavone, flavanone, isoflavones, flavanol, protocatechuic acid, resorcylic acid, gallic, caffeic acid, ferulic acid, chlorogenic acid, ascorbic acid, ascorbyl palmitate, carotenoids, cysteine hydrochloride, dithiothreitol, glutathione, thioglycolic acid, thiodipropionic acid, alpha-lipoic acid, and/or xanthines

39. The composition of claim 37 or 38, wherein the at least one additional anti-oxidizing compound is present at 0.1-5 wt %.

40. The composition of claim 37 or 38, wherein the at least one additional anti-oxidizing compound is present at 0.1-1 wt %.

41. The composition of any preceding claim, further comprising an anti-radical compound.

42. The composition of claim 41, wherein the anti-radical compound is present at 0.1-15 wt %.

43. The composition of claim 41, wherein the anti-radical compound is present at 0.1-1 wt %.

44. The composition of claim 41, wherein the anti-radical compound is present at 1-10 wt %.

45. The composition of claim 41, wherein the anti-radical compound is present at 6-8 wt %.

46. The composition of any preceding claim, wherein the at least one UV-filtering material is present at 10-35 wt %.

47. The composition of any of claims 1-45, wherein the at least one UV-filtering material is present at 0.1-15 wt %.

48. The composition of any of claims 1-45, wherein the at least one UV-filtering material is present at 0.1-5 wt %.

49. The composition of any of claims 1-45, wherein the at least one UV-filtering material is present at 0.1-1 wt %.

50. The composition of any preceding claim, wherein the composition comprises 0.1-1 wt % phenoxazone and/or phenoxazine compound, a precursor or derivative thereof, or a salt of any of the foregoing.

51. The composition of any of claims 1-49, wherein the composition comprises 0.01-0.1 wt % phenoxazone and/or phenoxazine compound, a precursor or derivative thereof, or a salt of any of the foregoing.

52. The composition of any of claims 1-49, wherein the composition comprises 0.01-0.05 wt % phenoxazone and/or phenoxazine compound, a precursor or derivative thereof, or a salt of any of the foregoing.

53. The composition of any of claims 1-49, wherein the composition comprises 0.1 wt % phenoxazone and/or phenoxazine compound, a precursor or derivative thereof, or a salt of any of the foregoing.

54. A method of maintaining the SPF of a composition comprising at least one UV-filtering material, comprising adding an unsaturated solution comprising 1 wt % or less of one or more phenoxazone and/or phenoxazine compound, a precursor or derivative thereof, or a salt of any of the foregoing to the composition to provide a final cosmetic formulation.

55. The method of claim 54, wherein the one or more phenoxazone and/or phenoxazine compound, a precursor or derivative thereof, or a salt of any of the foregoing comprises an xanthommatin, decarboxylated xanthommatin, uncyclized xanthommatin, ommatin D, dihydroxy-xanthommatin, or rhodommatin, or a precursor or derivative thereof, or a salt of any of the foregoing.

56. The method of claim 55, wherein the one or more phenoxazone and/or phenoxazine compound, a precursor or derivative thereof, or a salt of any of the foregoing comprises xanthommatin, or salt thereof.

57. The method of any of claims 54-56, wherein the at least one UV-filtering material comprises at least one UV-absorbing compound.

58. The method of claim 57, wherein the at least one UV-absorbing compound is selected from avobenzone, oxybenzone, oxybenzone cinoxate, homosalate, octisalate, octinoxate, octocrylene, and trolamine salicylate.

59. The method of any of claims 54-58, wherein the one or more phenoxazone and/or phenoxazine compound, a precursor or derivative thereof, or a salt of any of the foregoing comprises between 0.01-0.1 wt % in the final cosmetic formulation.

60. The method of any of claims 54-58, wherein the one or more phenoxazone and/or phenoxazine compound, a precursor or derivative thereof, or a salt of any of the foregoing comprises 0.01 wt % in the final cosmetic formulation.

61. The method of any of claims 54-60, wherein the SPF is maintained for at least one week.

62. The method of any of claims 54-60, wherein the SPF is maintained for at least two weeks.

63. The method of any of claims 54-60, wherein the SPF is maintained for at least three weeks.

64. A method of increasing the SPF of a composition comprising at least one UV-filtering material, comprising adding an unsaturated solution comprising 1 wt % or less of one or more phenoxazone and/or phenoxazine compound, a precursor or derivative thereof, or a salt of any of the foregoing to the composition to provide a final cosmetic formulation.

65. The method of claim 64, wherein the at least one UV-filtering material and the unsaturated solution of 1 wt % or less of one or more phenoxazone and/or phenoxazine compound, a precursor or derivative thereof, or a salt of any of the foregoing together exhibit synergy.

66. The method of claim 64 or 65, wherein the composition has an increase in pre-irradiation SPF of greater than 10% as compared to a composition comprising the at least one UV-filtering material without the unsaturated solution of 1 wt % or less of one or more phenoxazone and/or phenoxazine compound, a precursor or derivative thereof, or a salt of any of the foregoing.

67. The method of claim 64 or 65, wherein the composition has an increase in post-irradiation SPF of greater than 10% as compared to a composition comprising the at least one UV-filtering material without the unsaturated solution of 1 wt % or less of one or more phenoxazone and/or phenoxazine compound, a precursor or derivative thereof, or a salt of any of the foregoing.

68. The method of any of claims 64-67, wherein the one or more phenoxazone and/or phenoxazine compound, a precursor or derivative thereof, or a salt of any of the foregoing comprises an xanthommatin, decarboxylated xanthommatin, uncyclized xanthommatin, ommatin D, dihydroxy-xanthommatin, or rhodommatin, or a precursor or derivative thereof, or a salt of any of the foregoing.

69. The method of claim 68, wherein the one or more phenoxazone and/or phenoxazine compound, a precursor or derivative thereof, or a salt of any of the foregoing comprises xanthommatin, or salt thereof.

70. The method of any of claims 64-69, wherein the one or more phenoxazone and/or phenoxazine compound, a precursor or derivative thereof, or a salt of any of the foregoing comprises 0.03 wt % in the final cosmetic formulation.

71. The method of any of claims 64-70, wherein the at least one UV-filtering material comprises at least one UV-absorbing compound.

72. The method of claim 71, wherein the UV-absorbing compound comprises one of avobenzone, oxybenzone, oxybenzone cinoxate, homosalate, octisalate, octinoxate, octocrylene, and/or trolamine salicylate.

73. The method of any of claims 64-70, wherein the at least one UV-filtering material comprises at least one UV-scattering particle.

74. The method of claim 73, wherein the UV-scattering particle comprises titanium dioxide or zinc oxide.

75. An antioxidant composition comprising:
a vitamin E analogue; and
an unsaturated solution of one or more phenoxazone and/or phenoxazine compound, a precursor or derivative thereof, or a salt of any of the foregoing.

76. The composition of claim 75, wherein the composition performs as an antioxidant for longer than a composition comprising the vitamin E analogue alone.

77. The composition of claim 75 or 76, wherein the one or more phenoxazone and/or phenoxazine compound, a precursor or derivative thereof, or a salt of any of the foregoing comprises xanthommatin, decarboxylated xanthommatin, uncyclized xanthommatin, ommatin D, dihydroxy-xanthommatin, rhodommatin, or a precursor or derivative thereof, or a salt of any of the foregoing.

78. The composition of claim 77, wherein the one or more phenoxazone and/or phenoxazine compound, a precursor or derivative thereof, or a salt of any of the foregoing comprises xanthommatin, or a salt thereof.

79. The composition of any of claims 75-78, wherein the vitamin E analogue and the one or more phenoxazone and/or phenoxazine compound, a precursor or derivative thereof, or a salt of any of the foregoing, are present in a molar ratio of 1:1.

80. The composition of any of claims 75-79, wherein the vitamin E analogue is Trolox.

81. The composition of any of claims 75-80, comprising 0.01-1 wt % of the one or more phenoxazone and/or phenoxazine compound, a precursor or derivative thereof, or a salt of any of the foregoing.

82. The composition of any of claims 75-80, comprising 1-5 wt % of the one or more phenoxazone and/or phenoxazine compound, a precursor or derivative thereof, or a salt of any of the foregoing.

83. The composition of any of claims 75-80, comprising 1-10 wt % of the one or more phenoxazone and/or phenoxazine compound, a precursor or derivative thereof, or a salt of any of the foregoing.

84. The composition of any of claims 75-80, comprising 50 wt % of the one or more phenoxazone and/or phenoxazine compound, a precursor or derivative thereof, or a salt of any of the foregoing.

85. An antioxidant composition, comprising:
ascorbic acid; and
an unsaturated solution of one or more phenoxazone and/or phenoxazine compound, a precursor or derivative thereof, or a salt of any of the foregoing.

86. The composition of claim 86, wherein the composition performs as an antioxidant for longer than a composition comprising the ascorbic acid alone.

87. The composition of claim 85 or 86, wherein the one or more phenoxazone and/or phenoxazine compound, a precursor or derivative thereof, or a salt of any of the foregoing comprises xanthommatin, decarboxylated xanthommatin, uncyclized xanthommatin, ommatin D, dihydroxy-xanthommatin, rhodommatin, or a precursor or derivative thereof, or a salt of any of the foregoing.

88. The composition of claim 87, wherein the one or more phenoxazone and/or phenoxazine compound, a precursor or derivative thereof, or a salt of any of the foregoing comprises xanthommatin, or a salt thereof.

89. The composition of any of claims 85-88, wherein the ascorbic acid and the one or more phenoxazone and/or phenoxazine compound, a precursor or derivative thereof, or a salt of any of the foregoing are present in a molar ratio of 1:1 in a final formulation.

90. The composition of any of claims 85-89, comprising 0.01-1 wt % of the one or more phenoxazone and/or phenoxazine compound, a precursor or derivative thereof, or a salt of any of the foregoing.

91. The composition of any of claims 85-89, comprising 1-5 wt % of the one or more phenoxazone and/or phenoxazine compound, a precursor or derivative thereof, or a salt of any of the foregoing.

92. The composition of any of claims 85-89, comprising 1-10 wt % of the one or more phenoxazone and/or phenoxazine compound, a precursor or derivative thereof, or a salt of any of the foregoing.

93. The composition of any of claims 85-89, comprising 50 wt % of the one or more phenoxazone and/or phenoxazine compound, a precursor or derivative thereof, or a salt of any of the foregoing.
